SEQUENCE LISTING (45 sequences)

SEQ ID NO: 1 - DNA, 6176 bp, Mus musculus
  CDS (1)...(3756)
atggcgcgggcggacacgggccgcgggctcctggtgctgaccttctgcctgctgtccgcgcgcggggagctgccattgccccaggagacaactgtcaagctgagctgtgatgagggacccctgcaagtgatcctgggccctgagcaggctgtggtgctggactgcactttgggggctacagctgctgggcctccgaccagggtgacatggagcaaggatggagacactgtactagagcatgagaacctgcacctgctacccaatggctccctgtggctgtcctcacccctagagcaagaagacagcgatgatgaggaagctcttaggatctggaaggtcactgagggcagctattcctgtctggcccacagcccgctaggagtggtggccagccaggttgctgtggtcaagcttgccacactcgaagacttctctctgcaccccgagtcccagattgtggaggagaacgggacagcacgctttgaatgccacaccaagggccttccagcccccatcattacttgggaaaaggaccaggtgaccgtgcctgaggagccccggctcatcactcttcccaagtggctcctccagatcctagatgtccaggacagtgatgcaggctcctaccgctgcgtggccaccaattcagcccgccaacgattcagccaggaggcctcgctcactgtggccctcagagggtctttggaggctaccagggggcaggatgtggtcattgtggcagccccagagaacaccacggtagtgtctggacagaatgtagtgatggagtgcgtggcctctgctgaccccaccccttttgtgtcctgggtccgacaggatggaaagcctatctccacggatgtcatcgttctgggccggaccaatctactcatcgccagcgcgcagcctcggcactctggagtctatgtctgccgagccaacaagcccctcacgcgtgacttcgccactgcggctgctgagctccgagtgcttgctgccccagccatctcgcaggcacccgaggcgctctcgcggacgcgggccagcaccgcgcgcttcgtgtgccgggcgtccggggagccacggcccgcgctgcactggctgcacgacgggatcccgttgcgacccaatgggcgcgtcaaggtgcagggcggtggcggcagcttggtcatcactcagatcggcctgcaggacgctggctactaccagtgcgtagcagaaaacagcgcgggaactgcctgtgccgctgcgcccctggcggtagtggtgcgcgaggggctgcccagcgccccgactcgggtcacagccacgccgctgagcagctcctctgtgctggtggcctgggagcggcctgagttgcacagcgagcaaatcattggcttctctcttcactaccaaaaggcaaggggagtggacaatgtggagtaccagtttgcagtaaacaatgacaccacagagctgcaggttcgggacctggaacccaacacggattatgagttctacgtggtggcctactcccagctgggggccagccgaacctccagcccagccctggtgcatacactggacgatgtccccagcgcagcaccccagcttaccttgtccagccccaacccctcggacatcagggtggcatggctgcccctgccctccagcctgagcaatggacaggtgctgaagtacaagatagagtacggtttggggaaggaagatcaggttttctccaccgaggtgcctggaaatgagacacaacttacgttaaactcacttcagccaaacaaagtgtaccgagtccggatttcagctggcactggcgctggctatggagtcccttctcagtggatgcagcacaggacacctggtgtgcacaaccagagccatgttccctttgcccctgcagaattgaaggtgagggcaaagatggagtccctggtggtgtcatggcagccgccccctcaccccacccagatctctggatacaaactctactggggagaggtgggaacagaggaggaggcagatggtgaccgccccccagggggtcgtggagatcaagcttgggacgtcgggcccgtgcggctgaagaagaaagtgaagcagtatgaactgacccagttagtccctggcaggccgtacgaggtgaagctcgtagctttcaacaaacacgaggacggctacgctgctgtgtggaagggcaagacggagaaggcgcccacgccagacctgcctatccagagggggccaccgctgcctcctgcccatgtccacgcagagtcaaacagctccacttccatttggcttcggtggaagaagccagactttaccactgtcaagattgtcaactacactgtacgcttcggcccctgggggctcaggaatgcttccctggtcacctactataccagctctggagaagacattctcattggcggcctgaaaccatttaccaagtacgagtttgcggtacagtcccacggagtggatatggatgggccctttggctccgtcgtagaacgctccaccctgcctgaccggccttcaacacctccttctgacctgcgcctgagccccctgacaccatccaccgttcggttacactggtgtccccccacggagcccaatggtgagattgtggagtatctaattctctacagcaacaaccacacccagcccgaacaccagtggacactgctcaccacagagggaaacatcttcagtgcagaggtccatggcctagagagtgacactcggtatttcttcaagatgggagcccgcacagaggtggggcctgggcccttttcccgcttgcaggatgtgattactctgcaagagacattctcagactccttggatgtgcacgccgtcacgggcatcatcgtgggtgtctgcctgggccttctctgcctcctggcctgcatgtgtgctggcctacgacaaagctcccacagggaagcccttcccggattgtcctcctcaggcaccccaggaaacccagcgctctacacaagagctcggcttgggcctcccagtgtccctgctgcccatgagttggagtccctcgtgcatcctcgtccccaggattggtccccaccaccctcagatgtggaagacaaggctgaagtacacagccttatgggtggcagtgtttcagattgccggggccactccaagagaaagatctcctgggctcaggcagggggaccaaactgggcaggctcctgggcaggctgtgagctgccccagggtagtggtccaaggccggctctgacccgtgctctgctgcctccagcgggaaccgggcagacactgctgctgcaagccctggtgtatgacggcataaagagcaacgggagaaagaagccgtccccagcctgcaggaatcaggtggaagctgaggtcattgtccactccgacttcggtgcatccaaaggatgtcctgacctccacctccaagacctggagccagaggaaccactgactgcagagactctgccttccacgtctggagctgtggatctgtctcaaggagcagactggctgggcagggagctgggagggtgccaaccaacaaccagtgggccagagaggctcacctgcttgccagaagcagccagtgcctcctgctcctgctcagacctccagcccagcactgctatagaggaggcccctgggaaaagctgccagcccaaagccctgtgtcctctaacagtcagcccaagccttcccagggcccctgtctcctctgctcaggtcccctgagcagaaggcagatatggctcaggaacatgccatgcatggctacacatgtgtgtactagagatatccataagtccttggagcctcttagggtcctttggctggggttggggagaactttactctccctcatattctgcatcacatacaggagggacttgagacacagctctgtgtaatggacacgtgtgaagtcgtgtgtgtgtgtgtgtgtgtgtgtgctggttgagctaggaaacctctccctatgtagcactcactgtggcctagttgaccctccgtggcaggatggtgtaacagtgatcagtgccagctctttgagcttttagccttgtcacctagccttttattacactctgagagtgtctccagtgctgtgtctacaaagacagcgcccagccctcttctgtcagctgtgctgagcagagtgccagtcaactccacgggcctatgacaccgcagcctaccacagcatggctgtcatccccctggcctcctaaggtccagatgtctgggtgaacccagctcagctcccctctcctttgagcatctctgtacctaattttgtaatctgggaagtgcctggtttgggaaatcttctttcgcaccctgtccctctctgccccttccttcatttgttctggtgatctgtctcatgtcatcttgctcgattatcctggggcccttctctttcccatgatgcccctgatttcctcactgctgttttcatttctgtctgccatgcttgtctttatgtcgtgtgtttctcgtccctgagttcaacctatgcaccctttcctaacaacatgactacctcatgtctgcttcagaccatagtgtgacccctgggtccccacagctcccctgccaaccgccttcctgggcagatgagcccactccaagtagatctggaaaagacccttgtggcttgtctggctgccctccccttggtgttgagatgagaaggttttctatggaagagatgagtccaggctgcacaggggaacccccaagaaggggtagggagtgaaaccaagaggctgaaaaaaaatggctgccacccatctgcacagagagatgggtgtgtgcttttgacgtgcagtcctggctgaaactgaaggggtgaggagaggggagctactggggctgccatggctcagttccctgaccctggagccctgaacctggcttcagagtagcaaagagtttcctccaagatgctgtaagggaagtctttgcataggaaaagggcggctggctcattttattttatctttctttacactgaatcccaaaatcatcttaccacaaagggccaagcctgactggtatttcctgagtcacaagagccatgccatctctctggtttctcacctcagtcatgtcccagaattgtcagatccagtggcatctgtgctcttgctgcacatctttctatttcaactggctggcacatcaagtgttaactctggcttctgggccaagttagaaataaccagtctattttccctttattttattttattttattttattttatgtctttcagtggagttgtagcttctgaaagcgtctgtgtttattagccttgtgtgtcactcatgtttgaccccacccacatttccttctcctcccctcttcagccagcctatgataacactaaagattattaatgctggcttcgtatctcattaaagacaggattgtcacttgaactacttctatagcattcaaagtggccacggccaacaccaccgtatgtttcttcattgctctgaaggtcaagagcctcattttgttttcctggttagattcttttcctccttgccttgaatgaaataaccgttttaacagtaggctcttagcatcacaccacatagtcattcctcatgttcttgtttaacaagcacttgaggttctgggtttaaattaaatagctgcaaatgagacaatttataacccattaggctgggtggaaaattgttctcaaaagcaaataagtaataaatctggtatctgcctataactcacagttgataagaaagtagccagaactcactagcattatatatgattggggttctgagtaactggggagtgttagctttgtgactttgtagcaccaggtcttattaggaaagtctgttggccttttacagggcattagtccctttgtcgtttgccatggatgccttaagttctttggagtctcatttaagaattccttttctcgaagcatgacaagtgtatcgcaatacttacatgctcactcgtttacctggcttagtttgtgctgggttatttaattgcactttccagcatcatgcttcctccttacaaatatgatattctttattgttacactaaggtgttgatcatgtatctgtccctgtaaagaattaataaactattttccagac

SEQ ID NO: 2 - protein, 1252 aa, Mus musculus
MARADTGRGLLVLTFCLLSARGELPLPQETTVKLSCDEGPLQVILGPEQAVVLDCTLGATAAGPPTRVTWSKDGDTVLEHENLHLLPNGSLWLSSPLEQEDSDDEEALRIWKVTEGSYSCLAHSPLGVVASQVAVVKLATLEDFSLHPESQIVEENGTARFECHTKGLPAPIITWEKDQVTVPEEPRLITLPKWLLQILDVQDSDAGSYRCVATNSARQRFSQEASLTVALRGSLEATRGQDVVIVAAPENTTVVSGQNVVMECVASADPTPFVSWVRQDGKPISTDVIVLGRTNLLIASAQPRHSGVYVCRANKPLTRDFATAAAELRVLAAPAISQAPEALSRTRASTARFVCRASGEPRPALHWLHDGIPLRPNGRVKVQGGGGSLVITQIGLQDAGYYQCVAENSAGTACAAAPLAVVVREGLPSAPTRVTATPLSSSSVLVAWERPELHSEQIIGFSLHYQKARGVDNVEYQFAVNNDTTELQVRDLEPNTDYEFYVVAYSQLGASRTSSPALVHTLDDVPSAAPQLTLSSPNPSDIRVAWLPLPSSLSNGQVLKYKIEYGLGKEDQVFSTEVPGNETQLTLNSLQPNKVYRVRISAGTGAGYGVPSQWMQHRTPGVHNQSHVPFAPAELKVRAKMESLVVSWQPPPHPTQISGYKLYWGEVGTEEEADGDRPPGGRGDQAWDVGPVRLKKKVKQYELTQLVPGRPYEVKLVAFNKHEDGYAAVWKGKTEKAPTPDLPIQRGPPLPPAHVHAESNSSTSIWLRWKKPDFTTVKIVNYTVRFGPWGLRNASLVTYYTSSGEDILIGGLKPFTKYEFAVQSHGVDMDGPFGSVVERSTLPDRPSTPPSDLRLSPLTPSTVRLHWCPPTEPNGEIVEYLILYSNNHTQPEHQWTLLTTEGNIFSAEVHGLESDTRYFFKMGARTEVGPGPFSRLQDVITLQETFSDSLDVHAVTGIIVGVCLGLLCLLACMCAGLRQSSHREALPGLSSSGTPGNPALYTRARLGPPSVPAAHELESLVHPRPQDWSPPPSDVEDKAEVHSLMGGSVSDCRGHSKRKISWAQAGGPNWAGSWAGCELPQGSGPRPALTRALLPPAGTGQTLLLQALVYDGIKSNGRKKPSPACRNQVEAEVIVHSDFGASKGCPDLHLQDLEPEEPLTAETLPSTSGAVDLSQGADWLGRELGGCQPTTSGPERLTCLPEAASASCSCSDLQPSTAIEEAPGKSCQPKALCPLTVSPSLPRAPVSSAQVP

SEQ ID NO: 3 - DNA, 2796 bp, Mus musculus
  CDS (1)...(2796)
ggggagctgccattgccccaggagacaactgtcaagctgagctgtgatgagggacccctgcaagtgatcctgggccctgagcaggctgtggtgctggactgcactttgggggctacagctgctgggcctccgaccagggtgacatggagcaaggatggagacactgtactagagcatgagaacctgcacctgctacccaatggctccctgtggctgtcctcacccctagagcaagaagacagcgatgatgaggaagctcttaggatctggaaggtcactgagggcagctattcctgtctggcccacagcccgctaggagtggtggccagccaggttgctgtggtcaagcttgccacactcgaagacttctctctgcaccccgagtcccagattgtggaggagaacgggacagcacgctttgaatgccacaccaagggccttccagcccccatcattacttgggaaaaggaccaggtgaccgtgcctgaggagccccggctcatcactcttcccaagtggctcctccagatcctagatgtccaggacagtgatgcaggctcctaccgctgcgtggccaccaattcagcccgccaacgattcagccaggaggcctcgctcactgtggccctcagagggtctttggaggctaccagggggcaggatgtggtcattgtggcagccccagagaacaccacggtagtgtctggacagaatgtagtgatggagtgcgtggcctctgctgaccccaccccttttgtgtcctgggtccgacaggatggaaagcctatctccacggatgtcatcgttctgggccggaccaatctactcatcgccagcgcgcagcctcggcactctggagtctatgtctgccgagccaacaagcccctcacgcgtgacttcgccactgcggctgctgagctccgagtgcttgctgccccagccatctcgcaggcacccgaggcgctctcgcggacgcgggccagcaccgcgcgcttcgtgtgccgggcgtccggggagccacggcccgcgctgcactggctgcacgacgggatcccgttgcgacccaatgggcgcgtcaaggtgcagggcggtggcggcagcttggtcatcactcagatcggcctgcaggacgctggctactaccagtgcgtagcagaaaacagcgcgggaactgcctgtgccgctgcgcccctggcggtagtggtgcgcgaggggctgcccagcgccccgactcgggtcacagccacgccgctgagcagctcctctgtgctggtggcctgggagcggcctgagttgcacagcgagcaaatcattggcttctctcttcactaccaaaaggcaaggggagtggacaatgtggagtaccagtttgcagtaaacaatgacaccacagagctgcaggttcgggacctggaacccaacacggattatgagttctacgtggtggcctactcccagctgggggccagccgaacctccagcccagccctggtgcatacactggacgatgtccccagcgcagcaccccagcttaccttgtccagccccaacccctcggacatcagggtggcatggctgcccctgccctccagcctgagcaatggacaggtgctgaagtacaagatagagtacggtttggggaaggaagatcaggttttctccaccgaggtgcctggaaatgagacacaacttacgttaaactcacttcagccaaacaaagtgtaccgagtccggatttcagctggcactggcgctggctatggagtcccttctcagtggatgcagcacaggacacctggtgtgcacaaccagagccatgttccctttgcccctgcagaattgaaggtgagggcaaagatggagtccctggtggtgtcatggcagccgccccctcaccccacccagatctctggatacaaactctactggggagaggtgggaacagaggaggaggcagatggtgaccgccccccagggggtcgtggagatcaagcttgggacgtcgggcccgtgcggctgaagaagaaagtgaagcagtatgaactgacccagttagtccctggcaggccgtacgaggtgaagctcgtagctttcaacaaacacgaggacggctacgctgctgtgtggaagggcaagacggagaaggcgcccacgccagacctgcctatccagagggggccaccgctgcctcctgcccatgtccacgcagagtcaaacagctccacttccatttggcttcggtggaagaagccagactttaccactgtcaagattgtcaactacactgtacgcttcggcccctgggggctcaggaatgcttccctggtcacctactataccagctctggagaagacattctcattggcggcctgaaaccatttaccaagtacgagtttgcggtacagtcccacggagtggatatggatgggccctttggctccgtcgtagaacgctccaccctgcctgaccggccttcaacacctccttctgacctgcgcctgagccccctgacaccatccaccgttcggttacactggtgtccccccacggagcccaatggtgagattgtggagtatctaattctctacagcaacaaccacacccagcccgaacaccagtggacactgctcaccacagagggaaacatcttcagtgcagaggtccatggcctagagagtgacactcggtatttcttcaagatgggagcccgcacagaggtggggcctgggcccttttcccgcttgcaggatgtgattactctgcaagagacattctcagactccttggatgtgcac

SEQ ID NO: 4 - protein, 932 aa, Mus musculus
GELPLPQETTVKLSCDEGPLQVILGPEQAVVLDCTLGATAAGPPTRVTWSKDGDTVLEHENLHLLPNGSLWLSSPLEQEDSDDEEALRIWKVTEGSYSCLAHSPLGVVASQVAVVKLATLEDFSLHPESQIVEENGTARFECHTKGLPAPIITWEKDQVTVPEEPRLITLPKWLLQILDVQDSDAGSYRCVATNSARQRFSQEASLTVALRGSLEATRGQDVVIVAAPENTTVVSGQNVVMECVASADPTPFVSWVRQDGKPISTDVIVLGRTNLLIASAQPRHSGVYVCRANKPLTRDFATAAAELRVLAAPAISQAPEALSRTRASTARFVCRASGEPRPALHWLHDGIPLRPNGRVKVQGGGGSLVITQIGLQDAGYYQCVAENSAGTACAAAPLAVVVREGLPSAPTRVTATPLSSSSVLVAWERPELHSEQIIGFSLHYQKARGVDNVEYQFAVNNDTTELQVRDLEPNTDYEFYVVAYSQLGASRTSSPALVHTLDDVPSAAPQLTLSSPNPSDIRVAWLPLPSSLSNGQVLKYKIEYGLGKEDQVFSTEVPGNETQLTLNSLQPNKVYRVRISAGTGAGYGVPSQWMQHRTPGVHNQSHVPFAPAELKVRAKMESLVVSWQPPPHPTQISGYKLYWGEVGTEEEADGDRPPGGRGDQAWDVGPVRLKKKVKQYELTQLVPGRPYEVKLVAFNKHEDGYAAVWKGKTEKAPTPDLPIQRGPPLPPAHVHAESNSSTSIWLRWKKPDFTTVKIVNYTVRFGPWGLRNASLVTYYTSSGEDILIGGLKPFTKYEFAVQSHGVDMDGPFGSVVERSTLPDRPSTPPSDLRLSPLTPSTVRLHWCPPTEPNGEIVEYLILYSNNHTQPEHQWTLLTTEGNIFSAEVHGLESDTRYFFKMGARTEVGPGPFSRLQDVITLQETFSDSLDVH

SEQ ID NO: 5 - DNA, 825 bp, Mus musculus
  CDS (1)...(825)
cgacaaagctcccacagggaagcccttcccggattgtcctcctcaggcaccccaggaaacccagcgctctacacaagagctcggcttgggcctcccagtgtccctgctgcccatgagttggagtccctcgtgcatcctcgtccccaggattggtccccaccaccctcagatgtggaagacaaggctgaagtacacagccttatgggtggcagtgtttcagattgccggggccactccaagagaaagatctcctgggctcaggcagggggaccaaactgggcaggctcctgggcaggctgtgagctgccccagggtagtggtccaaggccggctctgacccgtgctctgctgcctccagcgggaaccgggcagacactgctgctgcaagccctggtgtatgacggcataaagagcaacgggagaaagaagccgtccccagcctgcaggaatcaggtggaagctgaggtcattgtccactccgacttcggtgcatccaaaggatgtcctgacctccacctccaagacctggagccagaggaaccactgactgcagagactctgccttccacgtctggagctgtggatctgtctcaaggagcagactggctgggcagggagctgggagggtgccaaccaacaaccagtgggccagagaggctcacctgcttgccagaagcagccagtgcctcctgctcctgctcagacctccagcccagcactgctatagaggaggcccctgggaaaagctgccagcccaaagccctgtgtcctctaacagtcagcccaagccttcccagggcccctgtctcctctgctcaggtcccc

SEQ ID NO: 6 - protein, 275 aa, Mus musculus
RQSSHREALPGLSSSGTPGNPALYTRARLGPPSVPAAHELESLVHPRPQDWSPPPSDVEDKAEVHSLMGGSVSDCRGHSKRKISWAQAGGPNWAGSWAGCELPQGSGPRPALTRALLPPAGTGQTLLLQALVYDGIKSNGRKKPSPACRNQVEAEVIVHSDFGASKGCPDLHLQDLEPEEPLTAETLPSTSGAVDLSQGADWLGRELGGCQPTTSGPERLTCLPEAASASCSCSDLQPSTAIEEAPGKSCQPKALCPLTVSPSLPRAPVSSAQVP

SEQ ID NO: 7 - DNA, 243 bp, Mus musculus
  CDS (1)...(243)
cctgagcaggctgtggtgctggactgcactttgggggctacagctgctgggcctccgaccagggtgacatggagcaaggatggagacactgtactagagcatgagaacctgcacctgctacccaatggctccctgtggctgtcctcacccctagagcaagaagacagcgatgatgaggaagctcttaggatctggaaggtcactgagggcagctattcctgtctggcccacagcccgctagga

SEQ ID NO: 8 - protein, 81 aa, Mus musculus
PEQAVVLDCTLGATAAGPPTRVTWSKDGDTVLEHENLHLLPNGSLWLSSPLEQEDSDDEEALRIWKVTEGSYSCLAHSPLG

SEQ ID NO: 9 - DNA, 192 bp, Mus musculus
  CDS (1)...(192)
gagaacgggacagcacgctttgaatgccacaccaagggccttccagcccccatcattacttgggaaaaggaccaggtgaccgtgcctgaggagccccggctcatcactcttcccaagtggctcctccagatcctagatgtccaggacagtgatgcaggctcctaccgctgcgtggccaccaattcagcccgc

SEQ ID NO: 10 - protein, 64 aa, Mus musculus
ENGTARFECHTKGLPAPIITWEKDQVTVPEEPRLITLPKWLLQILDVQDSDAGSYRCVATNSAR

SEQ ID NO: 11 - DNA, 189 bp, Mus musculus
  CDS (1)...(189)
tctggacagaatgtagtgatggagtgcgtggcctctgctgaccccaccccttttgtgtcctgggtccgacaggatggaaagcctatctccacggatgtcatcgttctgggccggaccaatctactcatcgccagcgcgcagcctcggcactctggagtctatgtctgccgagccaacaagcccctcacg

SEQ ID NO: 12 - protein, 63 aa, Mus musculus
SGQNVVMECVASADPTPFVSWVRQDGKPISTDVIVLGRTNLLIASAQPRHSGVYVCRANKPLT

SEQ ID NO: 13 - DNA, 195 bp, Mus musculus
  CDS (1)...(195)
cgggccagcaccgcgcgcttcgtgtgccgggcgtccggggagccacggcccgcgctgcactggctgcacgacgggatcccgttgcgacccaatgggcgcgtcaaggtgcagggcggtggcggcagcttggtcatcactcagatcggcctgcaggacgctggctactaccagtgcgtagcagaaaacagcgcggga

SEQ ID NO: 14 - protein, 65 aa, Mus musculus
RASTARFVCRASGEPRPALHWLHDGIPLRPNGRVKVQGGGGSLVITQIGLQDAGYYQCVAENSAG

SEQ ID NO: 15 - DNA, 249 bp, Mus musculus
  CDS (1)...(249)
agcgccccgactcgggtcacagccacgccgctgagcagctcctctgtgctggtggcctgggagcggcctgagttgcacagcgagcaaatcattggcttctctcttcactaccaaaaggcaaggggagtggacaatgtggagtaccagtttgcagtaaacaatgacaccacagagctgcaggttcgggacctggaacccaacacggattatgagttctacgtggtggcctactcccagctgggggccagc

SEQ ID NO: 16 - protein, 83 aa, Mus musculus
SAPTRVTATPLSSSSVLVAWERPELHSEQIIGFSLHYQKARGVDNVEYQFAVNNDTTELQVRDLEPNTDYEFYVVAYSQLGAS

SEQ ID NO: 17 - DNA, 249 bp, Mus musculus
  CDS (1)...(249)
agcgcagcaccccagcttaccttgtccagccccaacccctcggacatcagggtggcatggctgcccctgccctccagcctgagcaatggacaggtgctgaagtacaagatagagtacggtttggggaaggaagatcaggttttctccaccgaggtgcctggaaatgagacacaacttacgttaaactcacttcagccaaacaaagtgtaccgagtccggatttcagctggcactggcgctggctatgga

SEQ ID NO: 18 - protein, 83 aa, Mus musculus
SAAPQLTLSSPNPSDIRVAWLPLPSSLSNGQVLKYKIEYGLGKEDQVFSTEVPGNETQLTLNSLQPNKVYRVRISAGTGAGYG

SEQ ID NO: 19 - DNA, 288 bp, Mus musculus
  CDS (1)...(288)
tttgcccctgcagaattgaaggtgagggcaaagatggagtccctggtggtgtcatggcagccgccccctcaccccacccagatctctggatacaaactctactggggagaggtgggaacagaggaggaggcagatggtgaccgccccccagggggtcgtggagatcaagcttgggacgtcgggcccgtgcggctgaagaagaaagtgaagcagtatgaactgacccagttagtccctggcaggccgtacgaggtgaagctcgtagctttcaacaaacacgaggacggc

SEQ ID NO: 20 - protein, 96 aa, Mus musculus
FAPAELKVRAKMESLVVSWQPPPHPTQISGYKLYWGEVGTEEEADGDRPPGGRGDQAWDVGPVRLKKKVKQYELTQLVPGRPYEVKLVAFNKHEDG

SEQ ID NO: 21 - DNA, 246 bp, Mus musculus
  CDS (1)...(246)
ctgcctcctgcccatgtccacgcagagtcaaacagctccacttccatttggcttcggtggaagaagccagactttaccactgtcaagattgtcaactacactgtacgcttcggcccctgggggctcaggaatgcttccctggtcacctactataccagctctggagaagacattctcattggcggcctgaaaccatttaccaagtacgagtttgcggtacagtcccacggagtggatatggatggg

SEQ ID NO: 22 - protein, 82 aa, Mus musculus
LPPAHVHAESNSSTSIWLRWKKPDFTTVKIVNYTVRFGPWGLRNASLVTYYTSSGEDILIGGLKPFTKYEFAVQSHGVDMDG

SEQ ID NO: 23 - DNA, 252 bp, Mus musculus
  CDS (1)...(252)
acacctccttctgacctgcgcctgagccccctgacaccatccaccgttcggttacactggtgtccccccacggagcccaatggtgagattgtggagtatctaattctctacagcaacaaccacacccagcccgaacaccagtggacactgctcaccacagagggaaacatcttcagtgcagaggtccatggcctagagagtgacactcggtatttcttcaagatgggagcccgcacagaggtggggcctggg

SEQ ID NO: 24 - protein, 84 aa, Mus musculus
TPPSDLRLSPLTPSTVRLHWCPPTEPNGEIVEYLILYSNNHTQPEHQWTLLTTEGNIFSAEVHGLESDTRYFFKMGARTEVGPG

SEQ ID NO: 25 - DNA, 26 bp, Artificial Sequence
  oligonucleotide primer
aagcaggtgagcctctctggcccact

SEQ ID NO: 26 - DNA, 26 bp, Artificial Sequence
  oligonucleotide primer
cttgagacagatccacagctccagac

SEQ ID NO: 27 - DNA, 30 bp, Artificial Sequence
  oligonucleotide primer
atccgggaagggcttccctgtgggagcttc

SEQ ID NO: 28 - DNA, 26 bp, Artificial Sequence
  oligonucleotide primer
gcgctggggacatcgtccagtgtatg

SEQ ID NO: 29 - DNA, 28 bp, Artificial Sequence
  oligonucleotide primer
gttccaggtcccgaacctgcagctctgt

SEQ ID NO: 30 - DNA, 27 bp, Artificial Sequence
  oligonucleotide primer
ccactccccttgccttttggtagtgaa

SEQ ID NO: 31 - DNA, 21 bp, Artificial Sequence
  oligonucleotide primer
gtgctgaccttctgcctgctg

SEQ ID NO: 32 - DNA, 22 bp, Artificial Sequence
  oligonucleotide primer
ctctgtctgctacactggtcaa

SEQ ID NO: 33 - DNA, 18 bp, Artificial Sequence
  oligonucleotide primer
tggacgccaaggagttgg

SEQ ID NO: 34 - DNA, 19 bp, Artificial Sequence
  oligonucleotide primer
caaatcccacagaacagga

SEQ ID NO: 35 - DNA, 17 bp, Artificial Sequence
  oligonucleotide primer
acgggcatcatcgtggg

SEQ ID NO: 36 - DNA, 25 bp, Artificial Sequence
  oligonucleotide primer
gaggaggacaatccgggaagggctt

SEQ ID NO: 37 - DNA, 24 bp, Artificial Sequence
  oligonucleotide primer
tcaagcagttgacacttgactgtg

SEQ ID NO: 38 - DNA, 24 bp, Artificial Sequence
  oligonucleotide primer
taatctcacagtgatgagaggaga

SEQ ID NO: 39 - DNA, 26 bp, Artificial Sequence
  oligonucleotide primer
ctgtgtctcaatcttgaacaaacaca

SEQ ID NO: 40 - DNA, 25 bp, Artificial Sequence
  oligonucleotide primer
ggaagagagacagtaaacatttcgt

SEQ ID NO: 41 - DNA, 24 bp, Artificial Sequence
  oligonucleotide primer
ctcccttccttcctgatcgttttc

SEQ ID NO: 42 - DNA, 25 bp, Artificial Sequence
  oligonucleotide primer
cggctctcaagcactgcagattttg

SEQ ID NO: 43 - DNA, 500 bp, Mus musculus
  CDS (276)...(338)
aggctggtggcgcgcgggcgcgtgtcccctgtggtgcagggtggccacactggcggggcgcccccgcgtgggccgctagcccaagatggcgatggaggggcgggcgagctggccgcggccccggcccccgcgccggcccccgctcggccccggccccggaggcccgcgccccgcccgcggcgccgcgcctcccggagccactgacgcccggcgcgccctcccccggcggcggcccaggcgcccggacgcggcggcagcggcccgagcccggccctatggcgcgggcggacacgggccgcgggctcctggtgctgaccttctgcctgctgtccgcgcgcggtaagggcccgggtggccgcagtcgcgagtgggcgtccccggcgcccgcgatgcttgcgcgccgggggctgtggggacttgcccccagggggtgtgtgtccttgctgtgcacagcctggcaccgtgcgtgtccccctgcgcgtggcccttgtgcatgtgag

SEQ ID NO: 44 - protein, 21 aa, Mus musculus
MARADTGRGLLVLTFCLLSAR

SEQ ID NO: 45 - DNA, 3756 bp, Mus musculus
atggcgcgggcggacacgggccgcgggctcctggtgctgaccttctgcctgctgtccgcgcgcggggagctgccattgccccaggagacaactgtcaagctgagctgtgatgagggacccctgcaagtgatcctgggccctgagcaggctgtggtgctggactgcactttgggggctacagctgctgggcctccgaccagggtgacatggagcaaggatggagacactgtactagagcatgagaacctgcacctgctacccaatggctccctgtggctgtcctcacccctagagcaagaagacagcgatgatgaggaagctcttaggatctggaaggtcactgagggcagctattcctgtctggcccacagcccgctaggagtggtggccagccaggttgctgtggtcaagcttgccacactcgaagacttctctctgcaccccgagtcccagattgtggaggagaacgggacagcacgctttgaatgccacaccaagggccttccagcccccatcattacttgggaaaaggaccaggtgaccgtgcctgaggagccccggctcatcactcttcccaagtggctcctccagatcctagatgtccaggacagtgatgcaggctcctaccgctgcgtggccaccaattcagcccgccaacgattcagccaggaggcctcgctcactgtggccctcagagggtctttggaggctaccagggggcaggatgtggtcattgtggcagccccagagaacaccacggtagtgtctggacagaatgtagtgatggagtgcgtggcctctgctgaccccaccccttttgtgtcctgggtccgacaggatggaaagcctatctccacggatgtcatcgttctgggccggaccaatctactcatcgccagcgcgcagcctcggcactctggagtctatgtctgccgagccaacaagcccctcacgcgtgacttcgccactgcggctgctgagctccgagtgcttgctgccccagccatctcgcaggcacccgaggcgctctcgcggacgcgggccagcaccgcgcgcttcgtgtgccgggcgtccggggagccacggcccgcgctgcactggctgcacgacgggatcccgttgcgacccaatgggcgcgtcaaggtgcagggcggtggcggcagcttggtcatcactcagatcggcctgcaggacgctggctactaccagtgcgtagcagaaaacagcgcgggaactgcctgtgccgctgcgcccctggcggtagtggtgcgcgaggggctgcccagcgccccgactcgggtcacagccacgccgctgagcagctcctctgtgctggtggcctgggagcggcctgagttgcacagcgagcaaatcattggcttctctcttcactaccaaaaggcaaggggagtggacaatgtggagtaccagtttgcagtaaacaatgacaccacagagctgcaggttcgggacctggaacccaacacggattatgagttctacgtggtggcctactcccagctgggggccagccgaacctccagcccagccctggtgcatacactggacgatgtccccagcgcagcaccccagcttaccttgtccagccccaacccctcggacatcagggtggcatggctgcccctgccctccagcctgagcaatggacaggtgctgaagtacaagatagagtacggtttggggaaggaagatcaggttttctccaccgaggtgcctggaaatgagacacaacttacgttaaactcacttcagccaaacaaagtgtaccgagtccggatttcagctggcactggcgctggctatggagtcccttctcagtggatgcagcacaggacacctggtgtgcacaaccagagccatgttccctttgcccctgcagaattgaaggtgagggcaaagatggagtccctggtggtgtcatggcagccgccccctcaccccacccagatctctggatacaaactctactggggagaggtgggaacagaggaggaggcagatggtgaccgccccccagggggtcgtggagatcaagcttgggacgtcgggcccgtgcggctgaagaagaaagtgaagcagtatgaactgacccagttagtccctggcaggccgtacgaggtgaagctcgtagctttcaacaaacacgaggacggctacgctgctgtgtggaagggcaagacggagaaggcgcccacgccagacctgcctatccagagggggccaccgctgcctcctgcccatgtccacgcagagtcaaacagctccacttccatttggcttcggtggaagaagccagactttaccactgtcaagattgtcaactacactgtacgcttcggcccctgggggctcaggaatgcttccctggtcacctactataccagctctggagaagacattctcattggcggcctgaaaccatttaccaagtacgagtttgcggtacagtcccacggagtggatatggatgggccctttggctccgtcgtagaacgctccaccctgcctgaccggccttcaacacctccttctgacctgcgcctgagccccctgacaccatccaccgttcggttacactggtgtccccccacggagcccaatggtgagattgtggagtatctaattctctacagcaacaaccacacccagcccgaacaccagtggacactgctcaccacagagggaaacatcttcagtgcagaggtccatggcctagagagtgacactcggtatttcttcaagatgggagcccgcacagaggtggggcctgggcccttttcccgcttgcaggatgtgattactctgcaagagacattctcagactccttggatgtgcacgccgtcacgggcatcatcgtgggtgtctgcctgggccttctctgcctcctggcctgcatgtgtgctggcctacgacaaagctcccacagggaagcccttcccggattgtcctcctcaggcaccccaggaaacccagcgctctacacaagagctcggcttgggcctcccagtgtccctgctgcccatgagttggagtccctcgtgcatcctcgtccccaggattggtccccaccaccctcagatgtggaagacaaggctgaagtacacagccttatgggtggcagtgtttcagattgccggggccactccaagagaaagatctcctgggctcaggcagggggaccaaactgggcaggctcctgggcaggctgtgagctgccccagggtagtggtccaaggccggctctgacccgtgctctgctgcctccagcgggaaccgggcagacactgctgctgcaagccctggtgtatgacggcataaagagcaacgggagaaagaagccgtccccagcctgcaggaatcaggtggaagctgaggtcattgtccactccgacttcggtgcatccaaaggatgtcctgacctccacctccaagacctggagccagaggaaccactgactgcagagactctgccttccacgtctggagctgtggatctgtctcaaggagcagactggctgggcagggagctgggagggtgccaaccaacaaccagtgggccagagaggctcacctgcttgccagaagcagccagtgcctcctgctcctgctcagacctccagcccagcactgctatagaggaggcccctgggaaaagctgccagcccaaagccctgtgtcctctaacagtcagcccaagccttcccagggcccctgtctcctctgctcaggtcccc

(19) United States
(12) Patent Application Publication
(10) Pub. No.: US 2002/0102551 A1
(43) Pub. Date: Aug. 1, 2002

(54) Nope polypeptides, encoding nucleic acids and methods of use

(76) Inventor: J. Michael Salbaum, Bellevue, NE (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/754,997

(22) Filed: Jan. 4, 2001

Related U.S. Application Data
(60) Provisional application No. 60/174,496, filed on Jan. 4, 2000.
(60) Provisional application No. 60/205,789, filed on May 19, 2000.

(30) Foreign Application Priority Data
Oct. 26, 2000 (US) PCT/US00/29698

(51) Int. Cl. C12Q 1/68; G01N 33/53; C12P 21/02; C12N 5/06; C07K 16/18; C07K 14/435

(52) U.S. Cl. 435/6; 435/7.1; 435/69.1; 435/325; 435/320.1; 530/350; 530/389.1

(57) ABSTRACT

The invention provides an isolated Nope polypeptide, or functional fragment thereof, containing the amino acid sequence of a Nope polypeptide (SEQ ID NO:2), or a modification thereof. The invention also provides an isolated nucleic acid molecule encoding a Nope polypeptide amino acid sequence referenced as SEQ ID NO:2, or a modification thereof. The invention additionally provides an isolated nucleic acid molecule containing the nucleotide sequence referenced as SEQ ID NO:1, or a modification thereof. The invention further provides methods of detecting Nope polypeptides and Nope nucleic acid molecules.

This application claims the benefit of two U.S. Provisional Applications No. 60/174,496, filed Jan. 4, 2000, and No. 60/205,789, filed May 19, 2000, which are incorporated herein by reference.

### BACKGROUND OF THE INVENTION

The present invention relates generally to molecular genetics and, more specifically, to Nope polypeptides and encoding nucleic acid molecules.

All multi-celled organisms develop from a single cell by a complex process that coordinates the formation of particular tissues, structures and systems in the body to determine the morphology and function of the organism. The complex process of development of a single cell to a complex, multi-celled organism is regulated by the temporal and spatial expression of particular genes.

In complex, multi-celled organisms, the nervous system provides a network that allows the transmission of various signals from outside the organism to particular organs, tissues or systems, thereby allowing the organism to respond to external stimuli. The development of the nervous system requires the expression of developmentally regulated, tissue-specific genes that encode proteins required for the function of specific cell types that form the nervous system.

The formation of the nervous system in developing embryos requires the migration of specific types of cells. In the developing central nervous system, newly formed neurons migrate along predefined pathways to establish a variety of distinct structures within the adult brain. The formation of an axon, the long cellular process of a neuron, involves the navigation of the axon process to specific targets to establish the intricate networks of the central nervous system.

Axons function to propagate electrical signals between nerve cells and a target, for example, another nerve cell or a target tissue. In order to propagate the electrical signal to the proper target, the axon of the nerve cell must be guided during development to a particular target. During development of the nervous system, the guidance of the axons to particular targets is mediated by cell surface proteins that form specific ligand-receptor interactions.

A family of axon-associated adhesion receptors have been identified having a conserved structural motif, specifically an immunoglobulin domain, which resembles a structure found in immunoglobulins. These adhesion molecules are therefore classified as members of an immunoglobulin superfamily. The axon-associated adhesion receptors function to specifically bind to ligands and mediate cell-cell interactions in the developing nervous system. Although several axon-associated adhesion receptors have been identified, the identity of all axon guidance receptors that specifically function in guiding axons to their target and other gene products required for the development of the nervous system has not previously been determined.

Thus, there exists a need to identify genes that regulate the development of the nervous system and related biological functions. The present invention satisfies this need and provides related advantages as well.

### SUMMARY OF THE INVENTION

The invention provides an isolated Nope polypeptide, or functional fragment thereof, containing the amino acid sequence of a Nope polypeptide (SEQ ID NO:2), or a modification thereof. The invention also provides an isolated nucleic acid molecule encoding a Nope polypeptide amino acid sequence referenced as SEQ ID NO:2, or a modification thereof. The invention additionally provides an isolated nucleic acid molecule containing the nucleotide sequence referenced as SEQ ID NO:1, or a modification thereof. The invention further provides methods of detecting Nope polypeptides and Nope nucleic acid molecules.

### BRIEF DESCRIPTION OF THE DRAWINGS

FIG. 1 shows the genomic localization of the Nope gene, the tissue-specific expression of Nope mRNA, and the domain structure of Nope polypeptide.

FIG. 1A shows the location of expressed sequence tags (ESTs) in the genomic region upstream of the Punc gene, which are shown as black bars with the corresponding Genbank accession numbers indicated. The region designated e11 is the cloned restriction fragment used to generate a Nope hybridization probe. The Nope polyadenylation signal and the ATG start codon of the Punc gene are shown.

FIG. 1B shows the domain structure of the Nope protein in comparison to Neogenin, DCC, Punc, and NCAM.

FIG. 2 shows the nucleotide and amino acid sequence of Nope and the nucleotide sequence of Nope genomic DNA.

FIG. 2A shows the nucleotide sequence of the Nope cDNA (SEQ ID NO:1).

FIG. 2B shows the amino acid sequence derived from cDNA clones of the Nope gene (SEQ ID NO:2), which is encoded by nucleotides 1-3756 of FIG. 2a (SEQ ID NO:45). First shaded area corresponds to the signal peptide (amino acids 1-21); second shaded area corresponds to the transmembrane domain (amino acids 954-977); the first four underlined regions correspond to immunoglobulin (Ig) domains (Ig domain 1 (Ig1); amino acids 47-127)(Ig2; amino acids 155-218)(Ig3; amino acids 256-318)(Ig4; amino acids 347-411); the last five underlined regions correspond to fibronectin-type III (FnIII) domains (FnIII domain 1 (Fn1); amino acids 429-511)(Fn2; amino acids 527-609)(Fn3; amino acids 630-725)(Fn4; amino acids 750-831)(Fn5; amino acids 848-931).

FIG. 2C shows the nucleotide sequence of a genomic sequence (SEQ ID NO:43) encoding the 5′ region of the Nope cDNA. The start codon is shown in bold, the coding region of the first exon (SEQ ID NO:44) is underlined, and the splice site is shown in italics.

FIG. 3 shows the evolutionary relationships between Nope and other members of the Ig superfamily.

FIG. 3A shows the evolutionary relationship between Nope and the Ig superfamily.

FIG. 3B shows the evolutionary relationship between individual Ig domains derived from Nope, Punc, DCC, and Neogenin.

FIG. 3C shows the sequence relationship between Nope and Punc as shown by dot plot analysis based on a PAM similarity matrix. Sequence similarities appear as diagonal lines.

FIG. 4 shows chromosomal mapping of Nope to chromosome 9. Structures of the encoded proteins are indicated next to the chromosome sketch. Placement of Neogenin, Nope, Punc, and BAC end markers relative to framework markers D9Mit48 and D9Mit143 on chromosome 9 are shown. Distances are given in centiRays (cR). The arrangement of BAC clones and the origin of PCR products used for mapping is shown on the right.

### DETAILED DESCRIPTION OF THE INVENTION

The present invention provides Nope polypeptides and encoding nucleic acids. The invention also provides methods for detecting nucleic acids encoding Nope and methods for detecting Nope polypeptides. The methods of the invention are advantageous for specifically detecting the presence of a Nope polypeptide or a nucleic acid encoding Nope in a sample.

Nope is a newly identified mouse gene located on chromosome 9. As disclosed herein, the Nope polypeptide encoded by the Nope gene contains four immunoglobulin domains and five fibronectin-type III repeats, a single transmembrane domain and a cytoplasmic domain. Nope is a new member of the immunoglobulin superfamily of cell surface proteins and has a high level of similarity to Punc and to guidance receptors such as Deleted in Colorectal Cancer (DCC) and Neogenin. Nope is expressed during embryonic development in the notochord, in developing skeletal muscles, and later in the ventricular zone of the nervous system. In the adult brain, Nope is present in the hippocampus.

As used herein, the term “functional fragment,” when used in reference to a Nope polypeptide, is intended to refer to a portion of a Nope polypeptide that retains some or all or the activity of a Nope polypeptide. Exemplary functional fragments of a Nope polypeptide include the intracellular domain, the extracellular domain, the four individual immunoglobulin domains and the five individual fibronectin-type III domains. A functional domain contains an activity that is recognizable as a Nope polypeptide. For example, an intracellular domain contains the functional activity that mediates the signaling properties of the Nope polypeptide. The extracellular domain contains the functional activity of binding to a Nope ligand. The immunoglobulin and fibronectin domains are functional motifs that contribute to the structure and binding activity of the Nope extracellular domain.

In addition, other functional fragments of Nope are recognizable as providing a Nope polypeptide function. For example, a polypeptide fragment of Nope is recognizable as a functional fragment if the fragment can specifically bind to an antibody specific for a Nope polypeptide. Other functional fragments of a Nope polypeptide include Nope peptide fragments that are functional antigenic fragments, which can be used to generate a Nope-specific antibody.

As used herein, the term “polypeptide” when used in reference to Nope is intended to refer to a peptide or polypeptide of two or more amino acids. A “modification” of a Nope polypeptide can include a conservative substitution of the Nope amino acid sequence. Conservative substitutions of encoded amino acids include, for example, amino acids that belong within the following groups: (1) non-polar amino acids (Gly, Ala, Val, Leu, and Ile); (2) polar neutral amino acids (Cys, Met, Ser, Thr, Asn, and Gln); (3) polar acidic amino acids (Asp and Glu); (4) polar basic amino acids (Lys, Arg and His); and (5) aromatic amino acids (Phe, Trp, Tyr, and His). Other minor modifications are included within Nope polypeptides so long as the polypeptide retains some or all of its function as described herein.

A modification of a polypeptide can also include derivatives, analogues and functional mimetics thereof. For example, derivatives can include chemical modifications of the polypeptide such as alkylation, acylation, carbamylation, iodination, or any modification that derivatives the polypeptide. Analogues can include modified amino acids, for example, hydroxyproline or carboxyglutamate, and can include amino acids that are not linked by peptide bonds. Mimetics encompass chemicals containing chemical moieties that mimic the function of the polypeptide. For example, if a polypeptide contains two charged chemical moieties having functional activity, a mimetic places two charged chemical moieties in a spatial orientation and constrained structure so that the charged chemical function is maintained in three-dimensional space. Thus, a mimetic, which orients functional groups that provide a function of Nope, are included within the meaning of a Nope derivative. All of these modifications are included within the term “polypeptide” so long as the Nope polypeptide or functional fragment retains its function.

As used herein, the term “substantially” or “substantially the same” when used in reference to a nucleotide or amino acid sequence is intended to mean that the nucleotide or amino acid sequence shows a considerable degree, amount or extent of sequence identity when compared to a reference sequence. Such considerable degree, amount or extent of sequence identity is further considered to be significant and meaningful and therefore exhibit characteristics which are definitively recognizable or known. A substantially the same amino acid sequence retains comparable functional and biological activity characteristic of the reference polypeptide.

As used herein, the term “nucleic acid” means a polynucleotide such as deoxyribonucleic acid (DNA) or ribonucleic acid (RNA) and encompasses both single-stranded and double-stranded nucleic acid as well as an oligonucleotide. Nucleic acids useful in the invention include genomic DNA, cDNA and mRNA and can represent the sense strand, the anti-sense strand, or both. A genomic sequence of the invention includes regulatory regions such as promoters and enhancers that regulate Nope expression and introns that are outside of the exons encoding a Nope but does not include proximal genes that do not encode Nope. Exemplary Nope nucleic acids include the nucleotide sequence referenced as SEQ ID NOS:1 and 43, or fragments thereof. The term “isolated” in reference to a Nope nucleic acid molecule is intended to mean that the molecule is substantially removed or separated from components with which it is naturally associated, or otherwise modified by a human hand, thereby excluding Nope nucleic acid molecules as they exist in nature.

As used herein, the term “oligonucleotide” refers to a nucleic acid molecule that includes at least 15 contiguous nucleotides from a reference nucleotide sequence, and can include at least 16, 17, 18, 19, 20 or at least 25 contiguous nucleotides, and often includes at least 30, 40, 50, 60, 70, 80, 90, 100, 125, 150, 175, 200, 225, 250, 275, 300, 325, up to 350 contiguous nucleotides from the reference nucleotide sequence. The reference nucleotide sequence can be the sense strand or the anti-sense strand. The oligonucleotide can be chemically synthesized or expressed recombinantly.

As used herein, a “modification” of a nucleic acid can also include one or several nucleotide additions, deletions, or substitutions with respect to a reference sequence. A modification of a nucleic acid can include substitutions that do not change the encoded amino acid sequence due to the degeneracy of the genetic code. Such modifications can correspond to variations that are made deliberately, or which occur as mutations during nucleic acid replication. As such, a modification of a nucleic acid includes a substantially the same sequence, which is recognizable as a parent nucleic acid molecule such as the Nope nucleotide sequence referenced as SEQ ID NO:1. For example, a substantially the same nucleotide sequence can hybridize to the reference nucleotide sequence under moderately stringent or higher stringency conditions.

Exemplary modifications of the recited Nope sequences include sequences that correspond to homologs of other species such as human, primates, rat, rabbit, bovine, porcine, ovine, canine, feline or other animal species. The sequences of corresponding Nopes of non-mouse species can be determined by methods known in the art, such as by PCR or by screening genomic, cDNA or expression libraries.

Another exemplary modification of the recited Nope can correspond to splice variant forms of the Nope nucleotide sequence. Additionally, a modification of a nucleotide sequence can include one or more non-native nucleotides, having, for example, modifications to the base, the sugar, or the phosphate portion, or having a modified phosphodiester linkage. Such modifications can be advantageous in increasing the stability of the nucleic acid molecule.

Furthermore, a modification of a nucleotide sequence can include, for example, a detectable moiety, such as a radiolabel, a fluorochrome, a ferromagnetic substance, a luminescent tag or a detectable binding agent such as biotin. Such modifications can be advantageous in applications where detection of a Nope nucleic acid molecule is desired.

As used herein, a “vector” refers to a recombinant DNA or RNA plasmid or virus that comprises a polynucleotide. A vector can include an expression element operationally linked to a polynucleotide such that the expression element controls the expression of the polynucleotide. An “expression element” is a nucleotide sequence involved in an interaction of molecules that contributes to the functional regulation of a polynucleotide, including replication, transcription, splicing, translation, or degradation of the polynucleotide. An expression element that controls transcription of a gene can be a promoter, the site of initiation of transcription, or an enhancer, a DNA sequence that increases the rate of transcription.

As used herein, the term “sample” is intended to mean any biological fluid, cell, tissue, organ or portion thereof, that includes or potentially includes Nope nucleic acids or polypeptides. The term includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid or protein preparation. A sample can also be chemically synthesized, for example, by synthesizing degenerate oligonucleotides.

As used herein, the term “specifically hybridize” refers to the ability of a nucleic acid molecule to hybridize, under at least moderately stringent conditions or higher stringency conditions, as described herein, to a reference Nope nucleic acid molecule, without hybridization under the same conditions with nucleic acid molecules that are not Nope nucleic acid molecules, such as actin cDNA. Therefore, a nucleic acid molecule that specifically hybridizes to a Nope nucleic acid under high stringency conditions would not hybridize to a non-Nope nucleic acid under high stringency conditions.

The invention provides an isolated Nope polypeptide, or functional fragment thereof, comprising the amino acid sequence of a Nope polypeptide (SEQ ID NO:2), or a modification thereof. As disclosed herein, Nope was identified as a new member of the immunoglobulin superfamily that includes DCC, Neogenin and Punc.

Proteins of the immunoglobulin superfamily play essential roles in many biological functions of the surface of cells. This protein family is characterized by the presence of immunoglobulin (Ig) domains in the extracellular protein moiety and includes cell surface receptors for diffusible ligands as well as proteins that mediate cell adhesion (Brummendorf and Rathjen, Curr. Opin. Neurobiol. 6:584-593 (1996)). These proteins in general can act as signal transduction devices that can couple the presence of an extracellular cue to second messenger pathways inside the cell. While receptor tyrosine kinases and phosphatases can exert their influence on intracellular signaling directly (Holland et al., Curr. Opin. Neurobiol. 8:117-127 (1998)), there is evidence that classical cell adhesion molecules, which were originally thought to only provide mechanochemical linkage (Edelman and Crossin, Annu. Rev. Biochem. 60:155-190 (1991)), can associate with intracellular kinases (Maness et al., Perspect. Dev. Neurobiol. 4:169-181 (1996)) and thereby signal, for example, the presence of a favorable environment for cell migration. Many members of this protein family therefore play important roles in tissue formation and morphogenesis during embryonic development.

A subgroup of the Ig superfamily has been associated with migration and guidance of axonal growth cones during development of the vertebrate nervous system and is represented by the protein Deleted in Colorectal Cancer (DCC). Originally identified as a tumor suppressor (Fearon et al., Science 247:49-56 (1990)), DCC is a receptor for the axonal guidance cue Netrin-1 (Keino-Masu et al., Cell 87:175-285 (1996); Kennedy et al., Cell 78:425-435 (1994)). Members of this Ig superfamily subgroup are type I transmembrane proteins, with four Ig domains in their extracellular domain, and include frazzled in Drosophila (Kolodziej et al., Cell 87:197-204 (1996)), UNC-40 in C. elegans (Chan et al., Cell 87:187-195 (1996)), and Neogenin in vertebrates (Meyerhardt et al., Oncogene 14:1129-1136 (1997); Vielmetter et al., Genomics 41:414-421 (1997)).

DCC is characterized by the presence of four Ig domains in the extracellular domain. Other members of this Ig superfamily are present in invertebrate species, where frazzled and UNC-40 represent the DCC homologue in Drosophila and C. elegans, respectively. In vertebrates, DCC functions in the guidance of axonal growth cones and constitutes part of the receptor for the guidance cue Netrin-1 (Keino-Masu et al., supra, 1996; Kennedy et al., supra, 1994). Frazzled and UNC-40 perform a similar function in the respective invertebrate species (Chan et al., supra, 1996; Kolodziej et al., supra, 1996). Discovery of the gene for Neogenin (Meyerhardt et al., supra, 1997; Vielmetter et al., supra, 1997) revealed the existence of a second vertebrate homologue that can interact with Netrin-1 as well (Keino-Masu et al., supra, 1996). While the function of DCC in axon guidance has been confirmed in vitro (de la Torre et al., Neuron 19:1211-1124 (1997); Keino-Masu et al., supra, 1996) and in vivo (Fazeli et al., Nature 386:796-804 (1997)), the function of Neogenin is less clear, with implications that it can function as a guidance receptor for cell migration (Gad et al., supra, 1997; Keeling et al., Oncogene 15:691-700 (1997)).

A more distant member of the Ig superfamily subgroup that includes DCC is the mouse protein Punc (Salbaum, Mech. Dev. 71:201-204 (1998)). Punc was identified in a screen for genes regulated by the homeodomain transcription factor Is1-1 (Salbaum, supra, 1998; Karlsson et al., Nature 344:879-882 (1990)), which is essential for motor neuron development (Pfaff et al., Cell 84:309-320 (1996)). Punc has four Ig domains, as with other members of this family, but is a smaller protein with only two fibronectin-type III repeats in the extracellular domain, in contrast to six in other members of this family. Punc also differs in its regulation from other vertebrate members of this family. Both DCC and Neogenin have their onset of expression around mid-gestation in mice, increase their expression level, and expand in their expression domain during development (Gad et al., Dev. Biol. 192:258-273 (1997)). In contrast, Punc is expressed early after gastrulation in mouse embryos but undergoes a sharp down regulation after 11 days of gestation (Salbaum, supra, 1998). Expression of Punc is correlated with regions of proliferating cells, whereas DCC and Neogenin expression is in general associated with cells that have started to differentiate.

The down regulation of Punc is first evident in motor neurons of the spinal cord and constitutes an early step of motor neuron differentiation. Therefore, regulation of Punc and other cell surface receptors such as DCC and Neogenin demonstrates the role of transcriptional control in regulating cell surface properties, which can contribute to development and cell differentiation. To investigate the regulatory mechanism that controls the expression of the Punc gene, the genomic region encompassing the Punc gene and its upstream region was cloned (Salbaum, Genome 10:107-111 (1999)).

As disclosed herein, another gene was found located very close to the Punc gene, with a polyadenylation site not more that 3.5 kb from the ATG start codon of Punc (Example I). The newly identified gene close to Punc is a novel member of the immunoglobulin superfamily and, similar to Punc, belongs to the DCC subgroup. This newly identified gene is called Nope, for Neighbor of Punc ell, where ell is the probe used to identify the gene (see Example I).

Cloning genes of more distant members of the Ig subgroup such as Punc (Salbaum, supra, 1998) and Nope, as disclosed herein, suggested that the DCC Ig superfamily subgroup is more diverse than originally appreciated, and the subgroup can be referred to as the DEAL family or subgroup, for DCC et al. This diversity likely reflects recent events in the evolutionary history of vertebrates, since neither Punc nor Nope have a clear homologue in the nematode C. elegans (see Example III). The only protein sequence derived from the C. elegans genome that displays the characteristic four Ig domains is UNC-40, which is the homologue of DCC (Chan et al., supra, 1996). The presence of more than one gene of the DEAL subgroup is likely a recent acquisition in vertebrates.

The common structural motif of the DEAL proteins is the presence of four Ig domains, with the highest degree of similarity located in the fourth, innermost domain. It is likely that this domain is essential for extracellular interactions, in particular the binding of Netrin-1 to DCC or Neogenin (Gad et al., supra, 1997). All Ig domains display sequence features that classifies them as V type domains (Vaughn and Bjorkman, Neuron 16:261-273 (1996)). In contrast to the conserved number and sequence of the Ig domains, the number of FnIII repeats can vary and is diverse in the more distant members Punc and Nope (see Example III). Core members of the DEAL subgroup, for example, DCC, Neogenin, frazzled, and UNC-40, all have six FnIII repeats, whereas Nope has five and Punc has only two FnIII repeats. In addition to variation in the overall FnIII domain configuration, there is significant diversity in amino acid sequences. Together, the degree of conservation observed with the sequence and domain configuration suggests that the Ig domains are under higher selective pressure than the FnIII repeats. This supports the view that in DEAL proteins, as in other Ig CAMs (Brummendorf and Rathjen, supra, 1996), the biological interactions are executed via the Ig domain(s), whereas the FnIII repeats provide a structural function.

The cytoplasmic domains of Nope and Punc are also substantially distinct form core members of the DEAL family (see Example III). Both the Nope and the Punc sequence display no structural similarity to each other, to other cytoplasmic domains of the DEAL family, or to other protein domains or motifs in protein sequence databases. It has been demonstrated that DCC is engaged in multiple pathways of signal transduction, from interfacing with cAMP-dependent second messenger cascades during Netrin-1-dependent steering of axonal growth cones (Ming et al., Neuron 19:1225-1235 (1997)) to induction of apoptosis in the absence of the Netrin-1 ligand (Mehlen et al., Nature 395:801-804 (1998)). The structural correlate for these functions is thought to reside in the cytoplasmic domain. Due to the sequence divergence, it is likely that Nope-dependent signaling, for example, during myocyte differentiation, occurs through other proteins or pathways.

The invention provides an isolated Nope polypeptide, or functional fragment thereof. The isolated Nope polypeptides and peptides of the invention can be prepared by methods known in the art, including biochemical, recombinant and synthetic methods. For example, a Nope polypeptide can be purified by routine biochemical methods from a cell or tissue source that expresses the corresponding Nope transcript or polypeptide. The methods disclosed herein can be adapted for determining which cells and tissues, and which subcellular fractions therefrom, are appropriate starting materials. Biochemical purification can include, for example, steps such as solubilization of the appropriate tissue or cells, isolation of desired subcellular fractions, size, ion exchange or affinity chromatography, electrophoresis, and immunoaffinity procedures. The methods and conditions for biochemical purification of a polypeptide of the invention can be chosen by those skilled in the art, and purification monitored, for example, by an immunological assay or a functional assay.

The invention also provides a functional fragment of a Nope polypeptide. A functional fragment of a Nope polypeptide can be, for example, the extracellular domain of a Nope polypeptide, corresponding to amino acids 22-953 (SEQ ID NO:4). Additionally, a functional fragment can be the intracellular domain of a Nope polypeptide corresponding to amino acids 978-1252 (SEQ ID NO:6). The invention further provides a Nope polypeptide functional fragment comprising the amino acid sequence of immunoglobulin domain 1 (Ig1; amino acids 47-127; SEQ ID NO:8); immunoglobulin domain 2 (Ig2; amino acids 155-218; SEQ ID NO:10); immunoglobulin domain 3 (Ig3; amino acids 256-318; SEQ ID NO:12); immunoglobulin domain 4 (Ig4; amino acids 347-411; SEQ ID NO:14); fibronectin-type III domain 1 (Fn1; amino acids 429-511; SEQ ID NO:16); fibronectin-type III domain 2 (Fn2; amino acids 527-609; SEQ ID NO:18); fibronectin-type III domain 3 (Fn3; amino acids 630-725; SEQ ID NO:20); fibronectin-type III domain 4 (Fn4; amino acids 750-831; SEQ ID NO:22); or fibronectin-type III domain 5 (Fn5; amino acids 848-931; SEQ ID NO:24).

The invention also provides antibodies that specifically bind a Nope polypeptide. As used herein, the term “antibody” is used in its broadest sense to include polyclonal and monoclonal antibodies, as well as antigen binding fragments of such antibodies. With regard to an anti-Nope antibody of the invention, the term “antigen” means a native or synthesized Nope polypeptide or fragment thereof.

An anti-Nope antibody, or antigen binding fragment of such an antibody, is characterized by having specific binding activity for a Nope polypeptide or a peptide portion thereof of at least about 1×10⁵ M⁻¹. Thus, Fab, F(ab′)₂, Fd and Fv fragments of an anti-Nope antibody, which retain specific binding activity for a Nope polypeptide, are included within the definition of an antibody. Specific binding activity of a Nope polypeptide can be readily determined by one skilled in the art, for example, by comparing the binding activity of an anti-Nope antibody to a Nope polypeptide versus a control polypeptide that is not a Nope polypeptide. Methods of preparing polyclonal or monoclonal antibodies are well known to those skilled in the art (see, for example, Harlow and Lane, Antibodies: A Laboratory Manual, Cold Spring Harbor Laboratory Press (1988)). When using polyclonal antibodies, the polyclonal sera can be affinity purified using the antigen to generate mono-specific antibodies having reduced background binding and a higher proportion of antigen-specific antibodies.

In addition, the term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains as described by Huse et al. (Science 246:1275-1281 (1989)). These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Harlow and Lane, supra, 1988); Hilyard et al., Protein Engineering: A practical approach (IRL Press 1992); Borrabeck, Antibody Engineering, 2d ed. (Oxford University Press 1995)).

Anti-Nope antibodies can be raised using a Nope immunogen such as an isolated Nope polypeptide having the amino acid sequence of SEQ ID NO:2, or a fragment thereof, which can be prepared from natural sources or produced recombinantly, or a peptide portion of the Nope polypeptide. Such peptide portions of a Nope polypeptide are functional antigenic fragments if the antigenic peptides can be used to generate a Nope-specific antibody. A non-immunogenic or weakly immunogenic Nope polypeptide or portion thereof can be made immunogenic by coupling the hapten to a carrier molecule such as bovine serum albumin (BSA) or keyhole limpet hemocyanin (KLH). Various other carrier molecules and methods for coupling a hapten to a carrier molecule are well known in the art (see, for example, Harlow and Lane, supra, 1988). An immunogenic Nope polypeptide fragment can also be generated by expressing the peptide portion as a fusion protein, for example, to glutathione S transferase (GST), polyHis or the like. Methods for expressing peptide fusions are well known to those skilled in the art (Ausubel et al., Current Protocols in Molecular Biology (Supplement 47), John Wiley & Sons, New York (1999)).

The invention also provides a method of detecting a Nope polypeptide by contacting a sample with an antibody that specifically binds a Nope polypeptide and detecting specific binding of the antibody. An anti-Nope antibody is therefore useful, for example, for determining the presence or level of a Nope polypeptide in a sample.

An anti-Nope antibody is also useful for cloning a nucleic acid molecule encoding a gene encoding a polypeptide immunologically related to a Nope polypeptide from an appropriate expression library, for example, a lambda gt11 library. An anti-Nope antibody also can be used to substantially purify Nope from a sample, for example, from a cell extract of a cell or tissue expressing Nope or a cell extract from a cell expressing a Nope polypeptide from a recombinant nucleic acid molecule.

The invention also provides methods for detecting a Nope polypeptide in a sample by contacting the sample with an agent specific for Nope under conditions that allow specific binding of the agent to a Nope polypeptide and detecting the specifically bound agent. An agent specific for Nope is a molecule that specifically binds Nope. An example of a molecule that specifically binds Nope is a Nope antibody, or antigen binding fragment thereof. Additionally, the Nope binding and modulatory compounds identified in screening methods, as described below, are also suitable agents that can be used in methods of detecting Nope polypeptides.

Assays for detecting Nope polypeptides include, for example, immunohistochemistry, immunofluorescence, ELISA assays, radioimmunoassay, FACS analysis, immunoprecipitation, immunoblot analysis, and flow cytometry, using antibodies or antigen binding fragments specific for Nope (Harlow and Lane, supra, 1988; Harlow and Lane, Using Antibodies: A Laboratory Manual, Cold Spring Harbor Press (1999)). Various immunoassays are well known in the art, and can be readily modified by those skilled in the art in cases in which the agent is a Nope binding molecule other than an antibody. If desired, the agent or antibody can be rendered detectable by incorporation of, or by conjugation to, a detectable moiety, or binding to a secondary molecule that is itself detectable or detectably labeled.

A Nope polypeptide or an anti-Nope antibody can be labeled so as to be detectable using methods well known in the art (Hermanson, Bioconjugate Techniques, Academic Press, 1996; Harlow and Lane, supra, 1988). For example, the peptide or antibody can be labeled with various detectable moieties including a radiolabel, an enzyme, biotin or a fluorochrome. Reagents for labeling a peptide or antibody can be included in a kit containing the peptide or antibody or can be purchased separately from a commercial source. The invention further provides a kit, which contains a Nope polypeptide or an anti-Nope antibody or both. Such a kit also can contain a reaction cocktail that provides the proper conditions for performing an assay, for example, an ELISA or other immunoassay for determining the level of expression of a Nope polypeptide in a sample, and can contain control samples that contain known amounts of a Nope polypeptide and, if desired, a second antibody specific for the anti-Nope antibody. Where the kit is to be used for an immunoassay, it can include a simple method for detecting the presence or amount of a Nope polypeptide in a sample that is bound to the antibody.

The invention also provides an isolated nucleic acid molecule encoding a Nope polypeptide amino acid sequence referenced as SEQ ID NO:2, or a modification thereof. Such a nucleic acid molecule includes degenerate nucleotide sequences that encode the amino acid sequence referenced as SEQ ID NO:2. Additionally, the invention provides an isolated Nope nucleic acid molecule comprising the nucleotide sequence referenced as SEQ ID NO:1, or a modification thereof.

The invention additionally provides nucleic acid molecules having nucleotide sequences that encode functional fragments of a Nope polypeptide. For example, the invention provides a nucleotide sequence encoding the extracellular domain of a Nope polypeptide, corresponding to nucleotides 64-2859 (SEQ ID NO:3). Additionally, the invention provides a nucleotide sequence encoding the intracellular domain of a Nope polypeptide, corresponding to nucleotides 2932-3756 (SEQ ID NO:5). The invention further provides a nucleotide sequence encoding immunoglobulin domain 1 (Ig1; nucleotides 139-381; SEQ ID NO:7); immunoglobulin domain 2 (Ig2; nucleotides 463-654; SEQ ID NO:9); immunoglobulin domain 3 (Ig3; nucleotides 766-954; SEQ ID NO:11); immunoglobulin domain 4 (Ig4; nucleotides 1039-1233; SEQ ID NO:13); fibronectin-type III domain 1 (Fn1; nucleotids 1285-1533; SEQ ID NO:15); fibronectin-type III domain 2 (Fn2; nucleotides 1579-1827; SEQ ID NO:17); fibronectin-type III domain 3 (Fn3; nucleotides 1888-2175; SEQ ID NO:19); fibronectin-type III domain 4 (Fn4; nucleotides 2248-2493; SEQ ID NO:21); or fibronectin-type III domain 5 (Fn5; nucleotides 2542-2793; SEQ ID NO:23).

The invention also provides a modification of a Nope nucleotide sequence that hybridizes to a Nope nucleic acid molecule, for example, a nucleic acid molecule referenced as SEQ ID NO:1, under at least moderately stringent conditions. Modifications of Nope nucleotide sequences, where the modification has at least 60% identity to a Nope nucleotide sequence, are also provided. The invention also provides modification of a Nope nucleotide sequence having at least 65% identity, at least 70% identity, at least 75% identity, at least 80% identity, at least 85% identity, at least 90% identity, or at least 95% identity to a Nope nucleic acid such as that referenced as SEQ ID NO:1.

Moderately stringent conditions, as used herein, refers to hybridization conditions that permit a nucleic acid molecule to bind a nucleic acid that has substantial identity to a reference sequence. Moderately stringent conditions include conditions equivalent to hybridization of filter-bound nucleic acid in 50% formamide, 5× Denhart's solution, 5× SSPE, 0.2% SDS at 42° C., followed by washing in 0.2× SSPE, 0.2% SDS, at 42° C. In contrast, “highly stringent conditions” include conditions equivalent to hybridization of filter-bound nucleic acid in 50% formamide, 5× Denhart's solution, 5× SSPE, 0.2% SDS at 42° C., followed by washing in 0.2× SSPE, 0.2% SDS, at 65° C. Denhart's solution contains 1% Ficoll, 1% polyvinylpyrolidone, and 1% bovine serum albumin (BSA). 20× SSPE (sodium chloride, sodium phosphate, ethylene diamide tetraacetic acid (EDTA)) contains 3M sodium chloride, 0.2M sodium phosphate, and 0.025 M (EDTA). Other suitable moderately stringent and highly stringent hybridization buffers and conditions, including varying salt and temperature conditions, are well known to those of skill in the art and are described, for example, in Sambrook et al., Molecular Cloning: A Laboratory Manual, 2nd ed., Cold Spring Harbor Press, Plainview, New York (1989); and Ausubel et al., supra, 1999).

In general, a nucleic acid molecule that hybridizes to a recited sequence under moderately stringent conditions will have greater than about 60% identity, such as greater than about 70% identity or greater than about 80% identity to the reference sequence over the length of the two sequences being compared. A nucleic acid molecule that hybridizes to a recited sequence under highly stringent conditions will generally have greater than about 90% identity, including greater than about 95% identity, to the reference sequence over the length of the two sequences being compared. Identity of any two nucleic acid sequences can be determined by those skilled in the art based, for example, on a BLAST 2.0 computer alignment, using default parameters. BLAST 2.0 searching is available at http://www.ncbi.nlm.nih.gov/gorf/b12.html., as described by Tatiana et al., FEMS Microbiol Lett. 174:247-250 (1999).

The isolated Nope nucleic acid molecules of the invention can be used in a variety of diagnostic and therapeutic applications. For example, the isolated Nope nucleic acid molecules of the invention can be used as probes, as described above; as templates for the recombinant expression of Nope polypeptides; or in screening assays such as two-hybrid assays to identify cellular molecules that bind Nope.

The invention also provides isolated Nope oligonucleotides containing at least 15 contiguous nucleotides of the Nope nucleotide sequence referenced as SEQ ID NO:1, or the antisense strand thereof.

The Nope oligonucleotides of the invention that contain at least 15 contiguous nucleotides of a reference Nope nucleotide sequence are able to hybridize to Nope under moderately stringent or higher stringency hybridization conditions and thus can be advantageously used, for example, as probes to detect Nope DNA or RNA in a sample, and to detect splice variants thereof; as sequencing or PCR primers; as antisense reagents to block transcription of Nope RNA in cells; or in other applications known to those skilled in the art in which hybridization to a Nope nucleic acid molecule is desirable.

It is understood that a Nope nucleic acid molecule, as used herein, specifically excludes previously known nucleic acid molecules consisting of nucleotide sequences having identity with the Nope nucleotide sequence (SEQ ID NO:1), such as Expressed Sequence Tags (ESTs), Sequence Tagged Sites (STSs) and genomic fragments, deposited in public databases such as the nr, dbest, dbsts, gss and htgs databases, which are available for searching at http://www.ncbi.nlm.nih.gov/blast/blast.cgi?Jform=0, using the program BLASTN 2.0.9 described by Altschul et al., Nucleic Acids Res. 25:3389-3402 (1997).

In particular, a Nope nucleic acid molecule specifically excludes nucleic acid molecules consisting of any of the nucleotide sequences having the Genbank (gb), EMBL (emb) or DDBJ (dbj) Accession numbers set forth below: AW049847; AA051759; AA944556; AI154094; AI849335; AI599639; AA177505; AA403350; AA859434; AI429536; W33247; AA942729; AA389134; AV015396; AI046835; AW045411; AV047477; AA942730; AV016480; W83755; AL119290; AA253306; AI368698; D61677 (HUM430B04B); AI693740; AI101752; AA792362; AI339313; N53517; R24357; AV148364; AI653753; AA385851; AA947283; AI741225; AI599551; AI393663; T95325; AA706095; R69087; N53427; T89389; AA252900; R69201; AA086299; F09441 (HSC31F032); R36958; R36959; AA331887; R15778; AC021040 and AW109921.

The Nope nucleic acid molecules and oligonucleotides of the invention can be produced or isolated by methods known in the art (see, for example, Sambrook et al., supra, 1989; Ausubel et al., supra, 1999). The method chosen will depend, for example, on the type of nucleic acid molecule desired. Those skilled in the art, based on knowledge of the nucleotide sequences disclosed herein, can readily isolate Nope nucleic acid molecules as genomic DNA, or desired introns, exons or regulatory sequences therefrom; as full-length cDNA or desired fragments therefrom; or as full-length mRNA or desired fragments therefrom, by methods known in the art.

One useful method for producing a Nope nucleic acid molecule of the invention involves amplification of the nucleic acid molecule using PCR and Nope oligonucleotides. Either PCR or RT-PCR can be used to produce a Nope nucleic acid molecule having any desired nucleotide boundaries. Desired modifications to the nucleic acid sequence can also be introduced by choosing an appropriate oligonucleotide primer with one or more additions, deletions or substitutions. Such nucleic acid molecules can be amplified exponentially starting from as little as a single gene or mRNA copy, from any cell, tissue or species of interest.

The invention additionally provides a method of detecting a Nope nucleic acid molecule in a sample by contacting the sample with a Nope oligonucleotide under conditions allowing specific hybridization to a Nope nucleic acid molecule, and detecting specific hybridization. Also provided are methods for detecting a Nope nucleic acid molecule in a sample. The method consists of contacting the sample with a Nope nucleic acid molecule under conditions that allow specific hybridization to a Nope nucleic acid and detecting specific hybridization. The Nope nucleic acid molecule can be, for example, a Nope nucleotide sequence referenced as SEQ ID NO:1 or a Nope oligonucleotide containing at least 15 contiguous nucleotides of a reference Nope nucleotide sequence such as SEQ ID NO:1.

The invention additionally provides a method of detecting a Nope nucleic acid molecule in a sample by contacting the sample with two or more Nope oligonucleotides, amplifying a nucleic acid molecule, and detecting the amplification. The methods of detecting Nope nucleic acid in a sample can be either qualitative or quantitative, as desired. For example, the presence, abundance, integrity or structure of a Nope nucleic acid can be determined, as desired, depending on the assay format and the probe or primer pair chosen.

Useful assays for detecting a Nope nucleic acid based on specific hybridization with an isolated Nope nucleic acid molecule are well known in the art and include, for example, in situ hybridization, which can be used to detect altered chromosomal location of the nucleic acid molecule, altered gene copy number, and RNA abundance, depending on the assay format used. Other hybridization assays include, for example, Northern blots and RNase protection assays, which can be used to determine the abundance and integrity of different RNA splice variants, and Southern blots, which can be used to determine the copy number and integrity of DNA. A Nope hybridization probe can be labeled with any suitable detectable moiety, such as a radioisotope, fluorochrome, chemiluminescent marker, biotin, or other detectable moiety known in the art that is detectable by analytical methods.

Useful assays for detecting a Nope nucleic acid in a sample based on amplifying a Nope nucleic acid with two or more Nope oligonucleotides are also well known in the art, and include, for example, qualitative or quantitative polymerase chain reaction (PCR); reverse-transcription PCR (RT-PCR); single strand conformational polymorphism (SSCP) analysis, which can readily identify a single point mutation in DNA based on differences in the secondary structure of single-strand DNA that produce an altered electrophoretic mobility upon non-denaturing gel electrophoresis; and coupled PCR, transcription and translation assays, such as a protein truncation test, in which a mutation in DNA is determined by an altered protein product on an electrophoresis gel. Additionally, the amplified Nope nucleic acid can be sequenced to detect mutations and mutational hot-spots, and specific assays for large-scale screening of samples to identify such mutations can be developed.

The invention further provides a kit containing a Nope nucleic acid molecule, for example, a Nope nucleotide sequence referenced as SEQ ID NO:1 or a Nope oligonucleotide of the invention. For example, the diagnostic nucleic acids can be derived from any portion of SEQ ID NO:1 or an anti-sense strand thereof. Kits of the invention are useful as diagnostic systems for assaying for the presence or absence of nucleic acid encoding Nope in either genomic DNA, mRNA or cDNA.

A suitable diagnostic system includes at least one invention nucleic acid and can contain two or more invention nucleic acids as a separately packaged chemical reagent(s) in an amount sufficient for at least one assay. Instructions for use of the packaged reagent are also typically included. Those of skill in the art can readily incorporate invention nucleic acid probes and/or oligonucleotides useful as primers into kit form in combination with appropriate buffers and solutions for the practice of the invention methods as described herein.

The Nope nucleic acid molecules of the invention can be used to screen for nucleic acid molecules related to a Nope gene. Nucleic acid molecules related to Nope can be identified, for example, by screening a library, such as a genomic library, cDNA library or expression library, with a detectable agent. Such libraries are commercially available or can be produced from any desired tissue, cell, or species of interest using methods known in the art. For example, a cDNA or genomic library can be screened by hybridization with a detectably labeled nucleic acid molecule having a nucleotide sequence disclosed herein. Additionally, an expression library can be screened with an antibody raised against a polypeptide corresponding to the coding sequence of a Nope nucleic acid disclosed herein. The library clones containing Nope molecules of the invention can be isolated from other clones by methods known in the art and, if desired, fragments therefrom can be isolated by restriction enzyme digestion and gel electrophoresis.

The invention also provides a vector containing a Nope nucleic acid molecule. The vectors of the invention are useful for subcloning and amplifying a Nope nucleic acid molecule and for recombinantly expressing a Nope polypeptide. A vector of the invention can include, for example, viral vectors such as a bacteriophage, a baculovirus or a retrovirus; cosmids or plasmids; and, particularly for cloning large nucleic acid molecules, bacterial artificial chromosome vectors (BACs) and yeast artificial chromosome vectors (YACs). Such vectors are commercially available, and their uses are well known in the art.

The invention additionally provides a host cell containing a vector comprising a Nope nucleic acid molecule. Exemplary host cells that can be used to express recombinant Nope molecules include mammalian primary cells; established mammalian cell lines, such as COS, CHO, HeLa, NIH3T3, HEK 293 and PC12 cells; amphibian cells, such as Xenopus embryos and oocytes; and other vertebrate cells. Exemplary host cells also include insect cells such as Drosophila, yeast cells such as Saccharomyces cerevisiae, Saccharomyces pombe, or Pichia pastoris, and prokaryotic cells such as Escherichia coli.

The invention also provides methods of identifying cellular and non-cellular molecules that modulate Nope expression and activity. These molecules can be used, for example, in therapeutic applications to promote or inhibit a biological function of Nope.

As disclosed herein, the intracellular domain of the Nope polypeptide of the invention functions to mediate intracellular signaling. By specifically binding particular cellular proteins, the intracellular domain contributes to the function of Nope polypeptide, for example, in axonal guidance or proliferation of developing neurons. Such cellular proteins are themselves likely to have positive or negative effects on Nope activity, and are also appropriate targets for therapeutic intervention to prevent or treat disorders associated with aberrant Nope expression. Furthermore, peptides or analogs corresponding to the Nope binding interface of such cellular proteins, or of Nope, can be administered as therapeutic compounds to specifically interfere with Nope function.

Various binding assays to identify cellular proteins that interact with protein binding domains are known in the art and include, for example, yeast two-hybrid screening assays (see, for example, U.S. Pat. Nos. 5,283,173, 5,468,614 and 5,667,973; Ausubel et al., supra, 1999; Luban et al., Curr. Opin. Biotechnol. 6:59-64 (1995)) and affinity column chromatography methods using cellular extracts. By synthesizing or expressing polypeptide fragments containing various Nope sequences or deletions, the Nope binding interface can be readily identified.

The invention also provides a method of identifying non-cellular molecules, or Nope modulatory compounds, that modulate Nope expression or activity. A Nope modulatory compound is a molecule that specifically binds a Nope nucleic acid molecule or Nope polypeptide and alters its expression or activity. A Nope modulatory compound can be a naturally occurring macromolecule, such as a peptide or polypeptide, nucleic acid, carbohydrate, lipid, or any combination thereof. A Nope modulatory compound also can be a partially or completely synthetic derivative, analog or mimetic of such a macromolecule, or a small organic or inorganic molecule prepared partly or completely by combinatorial chemistry methods.

Methods for producing pluralities of compounds to use in screening for Nope modulatory compounds, including chemical or biological molecules such as simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources. Combinatorial libraries of molecules can be prepared using well known combinatorial chemistry methods (Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997)).

A variety of low- and high-throughput assays known in the art are suitable for detecting specific binding interactions between a Nope nucleic acid molecule or polypeptide and a candidate Nope modulatory compound. Both direct and competitive assays can be performed, including, for example, fluorescence correlation spectroscopy (FCS) and scintillation proximity assays (SPA) (reviewed in Major, J. Receptor Signal Transduction Res. 15:595-607 (1995); and in Sterrer et al., J. Receptor Signal Transduction Res. 17:511-520 (1997)). Assays for detecting specific binding interactions can include affinity separation methods using a Nope-specific ligand, for example, an antibody used in ELISA assays, FACS analysis or affinity separation.

Assays to identify compounds that modulate Nope gene expression can involve first transducing cells with a Nope promoter-reporter nucleic acid construct such that a change in expression of a protein such as β-lactamase, luciferase, green fluorescent protein or β-galactosidase will be detected in response to contacting the cell with a Nope modulatory compound that upregulates or downregulates expression of Nope. Such assays and reporter systems are well known in the art and are described, for example, in Ausubel et al., supra, 1999. Other assays to identify compounds that modulate Nope gene expression include assays that measure levels of Nope transcripts, such as Northern blots, RNase protection assays, and RT-PCR. Methods of identifying a Nope promoter and/or enhancer from Nope genomic DNA are well known in the art. A reporter gene construct can be generated using the promoter region of Nope and screened for compounds that increase or decrease Nope gene promoter activity. Such compounds can also be used to alter Nope expression.

Assays to identify compounds that modulate Nope polypeptide expression can involve detecting a change in Nope polypeptide abundance in response to contacting the cell with a Nope modulatory compound. Assays for detecting changes in polypeptide expression include, for example, immunoassays with specific Nope antibodies, such as immunoblotting, immunofluorescence, immunohistochemistry and immunoprecipitation assays.

Appropriate assays to determine whether a Nope modulatory compound inhibits or promotes Nope activity can be determined by those skilled in the art based on the biological activity of Nope as described below. For example, Nope can be screened with various compounds, as described above, to identify a Nope modulatory compound that alters expression of a Nope nucleic acid or polypeptide or that alters a biological activity of a Nope polypeptide.

The Nope polypeptides and nucleic acid molecules of the invention can be used in various diagnostic or therapeutic applications. The diagnostic and therapeutic applications can be based on various biological activities of Nope, as described herein. For example, the expression pattern of Nope, as disclosed herein, indicates that Nope can be involved in neurogenesis and proliferation control. Therefore, a Nope modulatory compound can be used to alter proliferative activity of Nope. The skilled artisan appreciates that molecular pathways involved in cell proliferation are generally well conserved among eukaryotic ogransisms. Therefore, a proliferation assay can be performed in any eukaryotic cell type in which altered proliferation can be detected including, for example, primary mammalian cells, normal and transformed mammalian cell lines, yeast, insect cells and amphibian cells. For example, a Nope nucleic acid can be transfected into a cell and a Nope polypeptide expressed recombinantly. The transfected cell containing Nope can be screened with various compounds, as described herein, to identify a Nope modulatory compound that alters a proliferative response of Nope.

As disclosed herein, Nope is homologous to DCC, which was initially thought to be a tumor suppressor because it is absent or reduced in expression in most late-stage human colon tumors (Kolodziej, Curr. Opin. Genet. Dev. 7:87-92 (1997). Alteration of DCC expression occurs late in tumor progression and is likely to be reduced during tumor progression. Inactivation of DCC also occurs in several other tumor types, including gastric, pancreatic, endometrial, breast, prostate, esophageal, bladder and squamous cell cancers (Fearon and Pierceall, Cancer Surveys 24:3-17 (1995)).

Based on the homology of Nope with DCC, it is possible that Nope can function as a tumor suppressor. If Nope can function as a tumor suppressor, the methods of the invention can be used as diagnostic methods to identify an individual predisposed to developing cancer, for example, by detecting reduced expression of a Nope nucleic acid molecule or Nope polypeptide by the methods disclosed herein. The diagnostic methods described herein can also be used to identify individuals at increased risk of developing a proliferative disease, such as cancer, due to hereditary mutations in a Nope gene.

Furthermore, if a Nope activity or loss thereof is associated with tumorigenesis, a tumor can be staged by determining changes in expression of a Nope nucleic acid molecule or polypeptide associated with a cancer. As such, the diagnostic methods described herein can also be adapted for use as prognostic assays. Such an application takes advantage of the observation that alterations in expression or structure of different tumor suppressor molecules can take place at characteristic stages in the progression of a proliferative disease or of a tumor. If a correlation can be determined between Nope expression and the stage of a tumor, such knowledge can be used by the clinician to select the most appropriate treatment for the tumor and to predict the likelihood of success of that treatment. One skilled in the art can readily determine a correlation between Nope expression and the stage of a tumor by measuring the expression of Nope at various stages of tumor development using the methods disclosed herein and determining such a correlation.

Bardet-Biedl syndrome is an autosomal recessive disorder characterized by mental retardation, obesity, polydactyly, retinitis pigmentosa and hypogonadism (Carmi et al., Human Mol. Gen. 4:9-13 (1995)). Patients with this disorder also have a high incidence of hypertension, diabetes mellitus, and renal and cardiovascular anomalies. As disclosed herein, the Nope gene is located on chromosome 15, and the 3′-untranslated region of the Nope gene showed sequence homology to two human STS marker, WI-18508 and WI-16786, which have been mapped close to a locus on chromosome 15 that is linked to Bardet-Biedl syndrome. Nope is expressed in the hippocampus, an area of the brain associated with cognitive functions such as learning and memory. Since Bardet-Biedl syndrome is associated with mental retardation, it is possible that altered Nope expression or activity, or altered expression or activity of a gene linked to Nope, can be associated with Bardet-Biedl syndrome. If an association between Nope or a Nope-linked gene and Bardet-Biedl syndrome is determined, the Nope nucleic acid molecules of the invention can be used to diagnose Bardet-Biedl syndrome.

In addition, a Nope nucleic acid molecule can be used in therapeutic methods to treat an individual having an altered Nope activity. An altered Nope activity that is decreased relative to normal Nope expression can be compensated, for example, by increasing expression of Nope by administering a nucleic acid encoding Nope. Accordingly, a decrease or loss of an activity associated with Nope can be compensated by administering a Nope nucleic acid in an expression vector that allows expression of a Nope polypeptide. Alternatively, an altered Nope activity that is increased relative to normal Nope expression can be decreased by administering a Nope anti-sense nucleic acid.

For example, loss of a tumor suppressor activity associated with Nope that has been decreased or eliminated in a tumor can be administered to the tumor to restore tumor suppressor activity. If altered Nope activity is associated with Bardet-Biedel syndrome, the Nope activity can also be altered by either increasing expression of Nope by introducing a nucleic acid encoding Nope or by decreasing expression of Nope using an anti-sense nucleic acid. A vector containing a Nope nucleic acid molecule can be introduced into an individual by in vivo or ex vivo methods to restore or increase expression of a Nope polypeptide. Vectors useful for such therapeutic methods include, for example, retrovirus, adenovirus, lentivirus, herpesvirus, poxvirus DNA or any viral DNA that allows expression of the heterologous polynucleotide of interest. Other vectors can also be employed, for example, DNA vectors, pseudotype retroviral vectors, adeno-associated virus, gibbon ape leukemia vector, vesicular stomatitis virus (VSV), VL30 vectors, liposome mediated vectors, and the like.

Nope modulatory compounds can also be used in therapeutic methods. For example, a Nope modulatory compound can be used to alter the expression or activity of a Nope polypeptide that is aberrantly expressed or has aberrant activity. For example, excessive proliferative activity associated with Nope can be reduced with a Nope modulatory compound that decreases expression of Nope or decreases Nope proliferative activity. If an altered Nope activity is associated with Bardet-Biedl syndrome, Nope modulatory compounds can be used to increase or decrease Nope activity, as appropriate, to treat signs or symptoms associated with Bardet-Biedl syndrome.

The present invention further provides transgenic non-human mammals that are capable of expressing exogenous nucleic acids encoding a Nope polypeptide. An exogenous nucleic acid refers to a nucleic acid sequence which is not native to the host, or which is present in the host in other than its native environment, for example, as part of a genetically engineered DNA construct. In addition to naturally occurring levels of Nope, a Nope polypeptide of the invention can either be overexpressed or underexpressed in transgenic mammals, for example, as in the well-known knock-out transgenics.

Also contemplated herein, is the use of homologous recombination of mutant or normal versions of Nope genes with the native gene locus in transgenic animals to alter the regulation of expression or the structure of Nope polypeptides by replacing the endogeneous gene with a recombinant or mutated Nope gene. Methods for producing a transgenic non-human mammal including a gene knock-out non-human mammal, are well known to those skilled in the art (see, Capecchi et al., Science 244:1288 (1989); Zimmer et al., Nature 338:150 (1989); Shastry, Experentia, 51:1028-1039 (1995); Shastry, Mol. Cell. Biochem., 181:163-179 (1998); and U.S. Pat. No. 5,616,491, issued Apr. 1, 1997, U.S. Pat. No. 5,750,826, issued May 12, 1998, and U.S. Pat. No. 5,981,830, issued Nov. 9, 1999).

Also provided are transgenic non-human mammals capable of expressing nucleic acids encoding a Nope polypeptide so mutated as to be incapable of normal activity and which, therefore, do not express native Nope. The present invention also provides transgenic non-human mammals having a genome comprising antisense nucleic acids complementary to nucleic acids encoding a Nope polypeptide, placed so as to be transcribed into antisense mRNA complementary to mRNA encoding a Nope polypeptide, which hybridizes to the mRNA and, thereby, reduces the translation thereof. The nucleic acid can additionally comprise an inducible promoter and/or tissue specific regulatory elements, so that expression can be induced, or restricted to specific cell types. Examples of nucleic acids are DNA or cDNA having a coding sequence substantially the same as the coding sequences shown in SEQ ID NO:1. An example of a non-human transgenic mammal is a transgenic mouse. Examples of tissue specificity-determining elements are the metallothionein promoter and the L7 promoter.

Animal model systems that elucidate the physiological and behavioral roles of Nope polypeptides are also provided and are produced by creating transgenic animals in which the expression of the Nope polypeptide is altered using a variety of techniques. Examples of such techniques include the insertion of normal or mutant versions of nucleic acids encoding a Nope polypeptide by microinjection, retroviral infection or other means well known to those skilled in the art, into appropriate fertilized embryos to produce a transgenic animal (see, for example, Hogan et al., Manipulating the Mouse Embryo: A Laboratory Manual (Cold Spring Harbor Laboratory, (1986)).

It is understood that modifications which do not substantially affect the activity of the various embodiments of this invention are also provided within the definition of the invention provided herein. Accordingly, the following examples are intended to illustrate but not limit the present invention.

### EXAMPLE I

### Cloning and Sequence Analysis of Nope

This example describes cloning and sequence analysis of the mouse Nope gene.

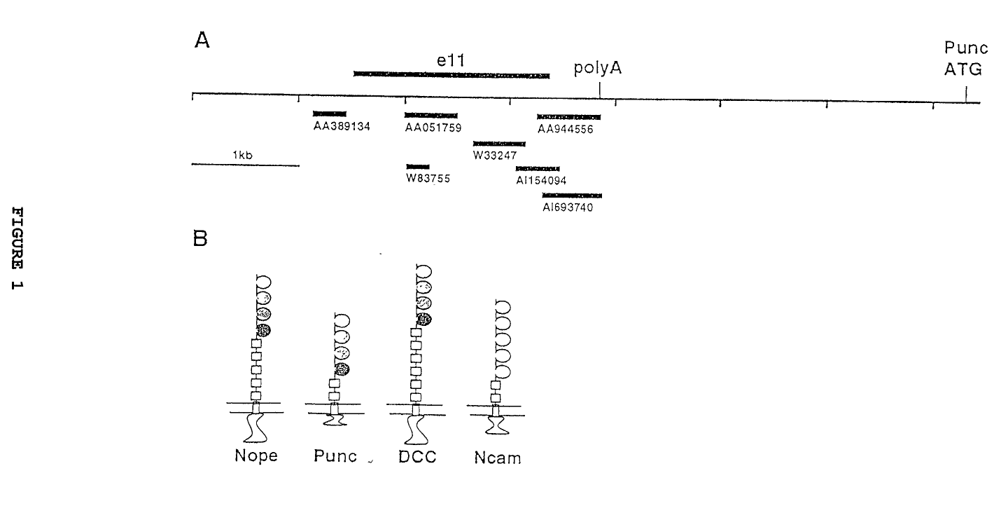

A novel mouse gene encoding a protein of the immunoglobin superfamily was identified by positional cloning on chromosome 9. In the course of experiments designed to understand the regulation of the mouse gene Punc, the DNA sequence of the genomic region starting at a BamHI site located 7.3 kb upstream of the Punc ATG start codon and ending at the second exon of Punc was determined. Analysis of the Punc 5′-upstream region revealed that a series of mouse, rat, and human ESTs were either identical or similar to genomic DNA sequences of the Punc gene locus (FIG. 1A). In FIG. 1A, the location of ESTs in the genomic region upstream of the Punc gene are depicted with black bars, and the corresponding Genbank Accession numbers are shown: mouse (AA389134, AA051759, W33247,W83755, AI154094); rat (AA944556); and human (AI693740). The ell region indicates the cloned restriction fragment used to generate a Nope hybridization probe. The Nope polyadenylation signal and the ATG start codon of the Punc gene are shown.

Analysis of the region 5′ of Punc revealed the presence of another gene close to the Punc gene. The polyadenylation site of the putative transcript was located 3.5 kb upstream of the Punc start codon. The transcriptional direction of the new gene was identical to that of Punc.

Using an EcoRV-Dra III restriction fragment from this region 5′ of Punc to generate an antisense riboprobe, hybridization experiments were carried out to determine whether this genomic region was transcribed. Briefly, for northern blot hybridization, RNA was extracted from adult mouse tissues using TRIZOL (Life Technologies; Gaithersburg Md.). Five μg RNA from each tissue was separated by gel electrophoresis, blotted onto a nylon membrane, and hybridized with a digoxigenin (DIG)-labeled (Boehringer Mannheim; Indianapolis Ind.) antisense riboprobe transcribed from a cloned 1.8 kb DraIII/EcoRV restriction fragment covering the 3′-untranslated area of the Nope gene (position 3819 to 5682). Hybridization signals were visualized by chemiluminescence.

Northern blot analysis on RNA samples prepared from adult mouse tissues revealed a single transcript of 6.5 kb detected by the probe derived from the 3′-untranslated region of the Nope gene. Nope expression was detected in brain, cerebellum, heart, and skeletal muscle. Nope expression was not detected in lung, liver, spleen, or kidney. These results indicated that a gene located upstream of the Punc gene is expressed and corroborated the EST data in public databases. Due to its genomic location, the novel gene was named Nope, which stands for Neighbor of Punc e11 (e11: probe plasmid).

Sequence analysis of the genomic fragment indicated the presence of a small reading frame without any discernible similarities to known protein sequences. Comparing the length of the putative 3′-untranslated region contained in the genomic sequence to the apparent length of the Nope mRNA as seen on the Northern blot revealed that the mRNA extended for about 3.8 kb in the 5′ direction. Cloning cDNA sequences that span this region was achieved with two steps of RACE (rapid amplification of cDNA ends) technology, using polyA⁺ RNA preparations from adult mouse muscle and from mouse embryos at 11.5 days of gestation.

Briefly, for cloning of cDNA derived from the Nope transcript, RNA was extracted from mouse embryos at 11.5 days of gestation or from adult mouse skeletal muscle tissue using TRIZOL (Life Technologies) and subjected to two cycles of oligo (dT) chromatography. cDNA was synthesized from 0.5 μg polyA⁺ RNA with Superscript II or Thermoscript reverse transcriptase (Life Technologies) and specific primers. cDNA was amplified by RACE or ligation-mediated anchor PCR procedures. The following oligonucleotide primers were used in two RACE steps together with reagents from RACE system (Life Technologies): cDNA synthesis step 1,5′-AAGCAGGTGAGCCTCTCTGGCCCACT-3′ (SEQ ID NO:25)(position 3599 in cDNA sequence); amplification 1,5′-CTTGAGACAGATCCACAGCTCCAGAC-3′ (SEQ ID NO:26)(position 3526); nested amplification 1,5′-ATCCGGGAAGGGCTTCCCTGTGGGAGCTTC-3′ (SEQ ID NO:27)(position 2965); cDNA synthesis step 2,5′-GCGCTGGGGACATCGTCCAGTGTATG-3′ (SEQ ID NO:28)(position 1583); amplification 2,5′-GTTCCAGGTCCCGAACCTGCAGCTCTGT-3′ (SEQ ID NO:29)(position 1480); nested amplification 2,5′-CCACTCCCCTTGCCTTTTGGTAGTGAA-3′ (SEQ ID NO:30)(position 1414). Amplification products were cloned (Invitrogen; Carlsbad Calif.) and sequenced using Thermo Sequenase (Amersham Pharmacia Biotech; Piscataway N.J.).

The sequence obtained covers a total of 6.1 kb, including the 3′-untranslated region (UTR). Attempts at obtaining further extension were unsuccessful. The difference in the obtained size of 6.1 kb compared to the apparent mRNA size of 6.5 kb was presumed to reside in sequences located further upstream as well as a polyadenosine tail. Conceptual translation of the cDNA sequence revealed an open reading frame of 1244 amino acids but no start codon.

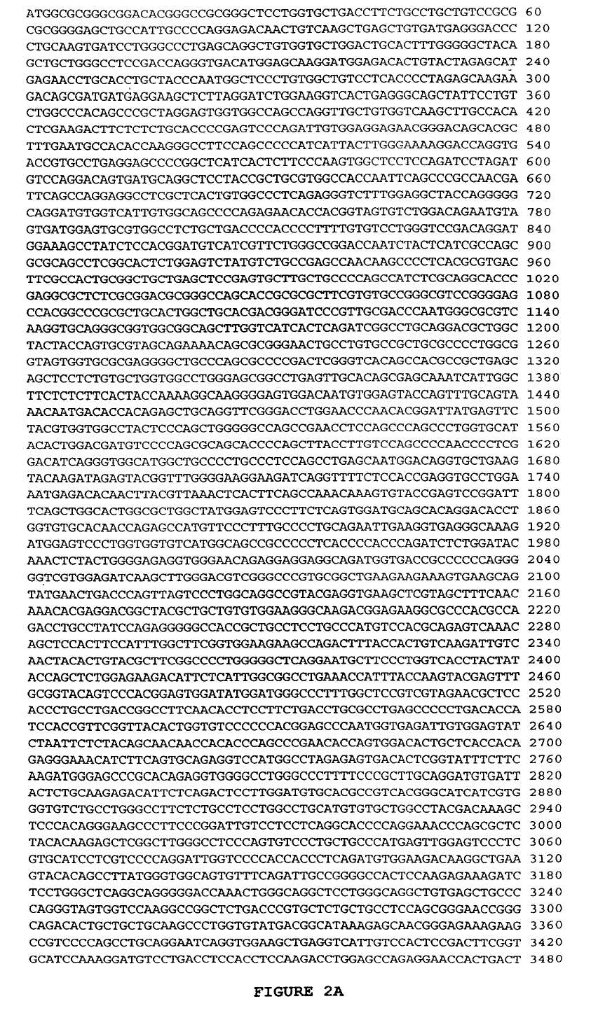

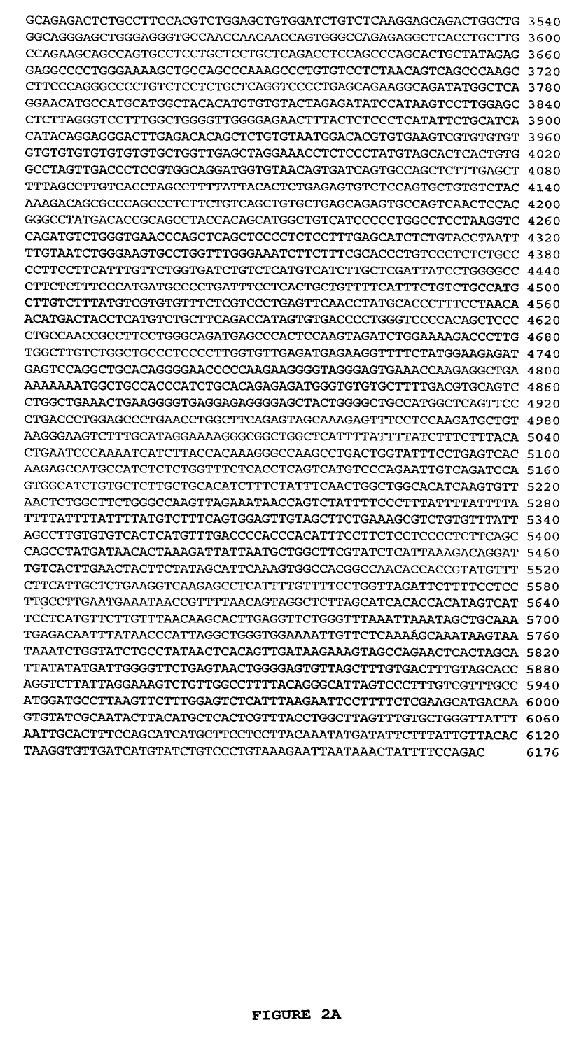

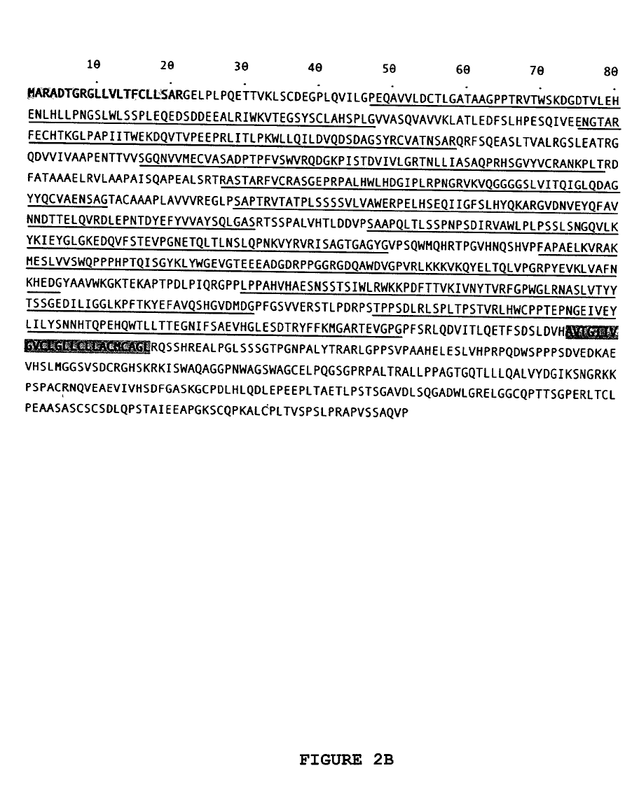

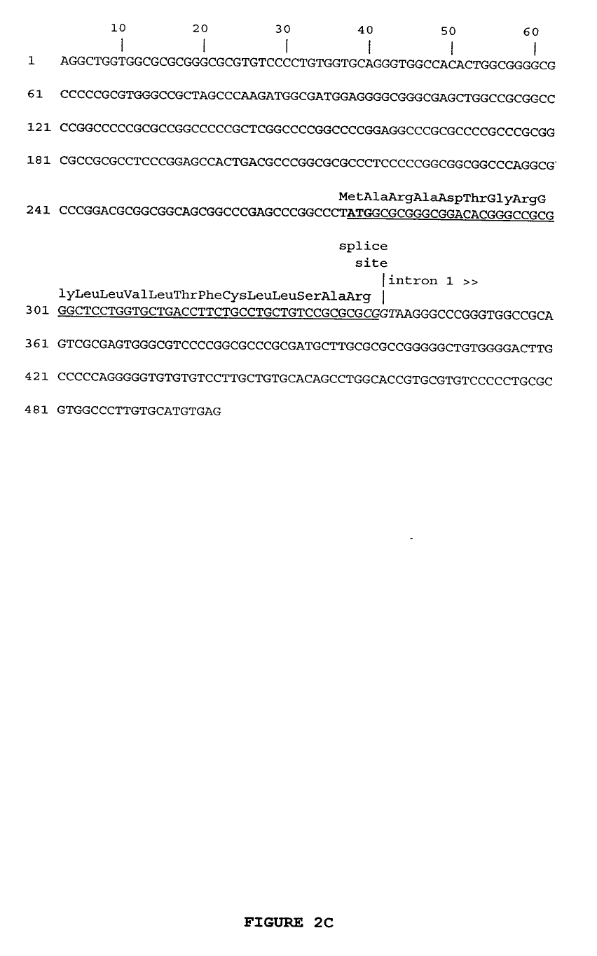

For genomic cloning, HindIII-StuI and HindIII-EcoRV restriction fragments from a BAC clone (Genome Systems; St. Louis Mo.) covering the Nope gene were identified by Southern blot hybridization with DIG-labeled (Boehringer) oligonucleotides 5′-GTGCTGACCTTCTGCCTGCTG-3′ (SEQ ID NO:31)(cDNA position 34) and 5′-CTCTGTCTGCTACACTGGTCAAC-3′ (SEQ ID NO:32)(located in the 3′-end of intron 1), cloned and sequenced. The cDNA sequence for the Nope mRNA is shown in FIG. 2A and is accessible under Genbank accession number AF176694. The sequence of genomic sequence encoding the first exon of Nope is shown in FIG. 2C.

Genomic cloning and sequencing of the relevant area from a BAC (bacterial artificial chromosome) clone demonstrated the presence of a single ATG codon 23 bp upstream of the starting base of the longest cDNA clone. The genomic sequence displays an extremely high GC content of 82% in 400 bp upstream of a splice donor site, possibly accounting for reverse transcriptase extension problems on this sequence.

Using the ATG identified in the genomic clone as the start codon for translation yielded a novel protein sequence of 1252 amino acids (FIG. 2B). Protein domain analysis of the Nope Sequence revealed the presence of a signal peptide, four immunoglobulin (Ig) domains, five fibronectin-type III (FnIII) repeats, a transmembrane domain and a cytoplasmic domain of 274 amino acids (see FIG. 1B). FIG. 1C shows the domain structure of the Nope protein in comparison to DCC, Punc, and NCAM. Shading indicates similarity among Ig domains. The domain structure indicates that the Nope protein most closely resembles cell adhesion molecules.

These results show that a novel gene, termed Nope, is a member of the immunoglobulin superfamily and is structurally related to cell adhesion molecules, in particular Punc and DCC.

### EXAMPLE II

### Developmental Expression of Nope mRNA

This example describes the tissue distribution and developmental expression of Nope mRNA as detected by in situ hybridization.

To determine the tissue distribution and developmental expression of Nope, in situ hybridization was performed on developing mouse embryos. Briefly, in situ hybridization was performed using DIG-labeled antisense riboprobes on 25 μm cryosections prepared from frozen tissues essentially as described previously (Long and Salbaum, Mol. Biol. Evol. 15:284-292 (1998)). A probe for myosin heavy chain was derived form an EST (Genbank accession number AF200922).

In situ hybridization analyses with a Nope antisense riboprobe demonstrated that Nope is expressed in the developing mouse embryo. The first weak signals of Nope expression can be detected in the notochord at 9.5 days of gestation (E9.5). In a cross section through the cervical region of a mouse embryo at E10.5, the main expression domain of Nope is in developing muscle tissues, starting in the dermomyotome of somites at E10.5 and is evident in the developing muscles of the forelimb and the body wall. A section through the cervical region of a mouse embryo at E15.5 revealed expression in skeletal muscles and in the nervous system. Nope expression in notochord is still visible at E10.5. Nope expression increases with the growth of muscles. In a cervical section at E15.5, hybridization signals for Nope can be found in all skeletal muscles, similar to a hybridization signal for the embryonic form of myosin heavy chain. In addition to muscle-specific expression, a Nope signal can be detected at E15.5 in the developing nervous system. A coronal section through the head of an embryo at E15.5 revealed Nope expression in the ventricular zone of the forebrain. Nope expression was observed in the developing muscles at E15.5 with a perinuclear localization of the transcript, which resulted in a “doughnut”-shaped appearance of the staining on cross sections. Nope expression is concentrated in the ventricular zone in the brain, a region containing proliferating neuroblasts as well as developing glial cells. In a section of an adult brain, Nope expression can be found in the hippocampus, the piriform cortex, thalamic nuclei, and foliae of the cerebellum. This expression pattern suggests that Nope initially contributes to the cell surface properties of developing muscle cells and functions in cells of the nervous system that arise late in gestation.

These results show that Nope expression increases during development and that Nope is expressed in the notochord, developing muscle tissue and in specific regions of the brain, including the ventricular zone, hippocampus, piriform cortex, thalamic nuclei and cerebellum.

### EXAMPLE III

### Evolutionary Relationship Between Nope and Immunoglobulin Superfamily Members

This example describes the evolutionary relationship between Nope and various members of the immunoglobulin superfamily.

To characterize the relationship between Nope and other known sequences, protein database searches were performed to analyze sequence relationships between Nope and other proteins. Sequence analysis and assembly was performed using MacMolly software (SoftGene; Berlin, Germany). On-line database searches were performed using BLAST (Altschul et al., Nucleic Acids Res. 25:3389-3402 (1997)), and domain analysis of the Nope protein sequence was carried out using SMART (Schultz et al., Proc. Natl. Acad. Sci. USA 95:5857-5864 (1998)). Signal peptides were predicted using the program SignalP (Nielsen et al., Protein Engineering 10:1-6 (1997)).

Analysis of the evolutionary relationships between the immunoglobulin domains of Nope and related proteins of the immunoglobulin superfamily was done with the program PAUP (Sinauer Associates; Sunderland Mass.). Alignments were constructed either from regions spanning a total of four Ig domains or from sequences representing individual single Ig domains. In the case of NCAM and L1, the four Ig domains closest to the FnIII repeats were selected. As outgroup in this analysis, a heavy chain variable domain sequence derived from an antibody against hepatitis B Virus X protein was chosen (Genbank Accession No. AAC82376). Dot plot representations of sequence comparisons between Nope and Punc are based on PAM matrix and were constructed (match length 20, up to 8 mismatches) using MacMolly Complign software (SoftGene).

Nope is most similar to Punc, with 45% sequence identity in the region ranging from the beginning of the second Ig domain throughout the first FnIII repeat. Other protein sequences that were similar to Nope are Neogenin (Meyerhardt et al., Oncogene 14:1129-1136 (1997); Vielmetter et al., Genomics 41:414-421 (1997)) and DCC (Fearon et al., Science 247:49-46 (1990)). Nope therefore belongs to a subfamily of the immunoglobulin superfamily of proteins that is characterized by the presence of four Ig domains and consists of vertebrate DCC, Neogenin, and Punc, Drosophila frazzled, and UNC-40 of C. elegans. DCC, frazzled and UNC-40 can all function as axonal guidance receptors (Chan et al., Cell 87:187-195 (1996); Keino-Masu et al., Cell 87:175-185 (1996); Kolodziej et al., Cell 87:197-204 (1996)). Therefore, Nope likely has a similar function as an axonal guidance receptor.

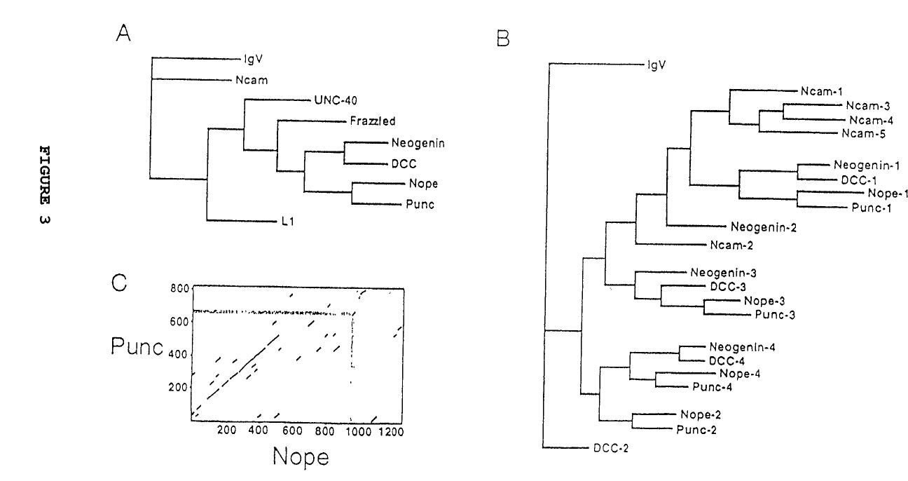

Analysis of sequence relationships of Nope, Punc, and other guidance receptors or neuronal cell adhesion molecules demonstrated that both Nope and Punc are more similar to the DCC subgroup of the Ig superfamily than they are to classical neuronal cell adhesion molecules such as NCAM (Cunningham et al., Science 236:799-806 (1987)) or L1 (Kobayasi et al., Biochem. Biophys. Acta 1090:238-240 (1991))(FIG. 3A). Within this branch, Nope and Punc group closely together, as do DCC and Neogenin, whereas sequences from invertebrate species appear to be more distantly related.

Examination of individual Ig domains from vertebrate protein sequences revealed that, within the DCC subgroup, cognate domains from different proteins are more similar than adjacent domains within the same molecule (FIG. 3B), as demonstrated by the grouping together of the first, third, and fourth Ig domains of DCC, Neogenin, Nope and Punc. Within these branches, relationships are conserved, since DCC and Neogenin sequences are closely related, as are sequences from Nope and Punc. In contrast, domains from the neural cell adhesion molecule NCAM are more similar to each other than they are to domains from proteins of the DCC subgroup.

While the close relationship between Nope and Punc could be clearly demonstrated for large parts of the extracellular domain, the close relationship does not extend to the whole protein sequence (FIG. 3C). FIG. 3C shows the sequence relationship between Nope and Punc as analyzed by dot plot analysis based on a PAM similarity matrix. Only the region containing the Ig domains and the first adjacent FnIII repeat are highly similar between the two proteins. The respective cytoplasmic domains of Nope and Punc did not exhibit sequence similarity to each other, as indicated by the lack of diagonal lines in this region, and database searches did not yield any other protein motif or domain matching Nope or Punc. Therefore, Nope is related to guidance receptors of the DCC family and can participate in similar extracellular interactions while containing a novel, diverse intracellular signaling domain.

The human gene for Neogenin has previously been mapped to chromosome 15 in the band 15q22.3-23 (Meyerhardt et al., supra, 1997; Vielmetter et al., supra, 1997), and human PUNC has been placed on the same band of chromosome 15 (Salbaum, Mamm. Genome 10:107-111 (1999)). To determine whether a similar colocalization exists in the mouse and to obtain high resolution positional information for the Nope gene, Neogenin, Nope, Punc, and two BAC end sequences were mapped using radiation hybrid mapping on the mouse T31 panel (McCarthy et al., Genome Res. 7:1153-1161 (1997)).

Briefly, for radiation hybrid mapping, DNA from the T31 radiation hybrid panel (McCarthy et al., supra, 1997) was obtained from Research Genetics and used for mapping experiments. The following oligonucleotide primer pairs were used: Punc (from genomic DNA sequence), 5′-TGGACGCCAAGGAGTTGG-3′ (SEQ ID NO:33), and 5∝-CAAATCCCACAGAACAGGA-3′ (SEQ ID NO:34), amplicon size 1236 bp; Nope (upstream primer derived from genomic DNA, downstream primer from cDNA sequence at position 2975), 5′-ACGGGCATCATCGTGGG-3′ (SEQ ID NO:35) and 5′-GAGGAGGACAATCCGGGAAGGGCTT-3′ (SEQ ID NO:36), amplicon size 592 bp; Neogenin (from cDNA genbank Acc. No. Y09535, position 5107 to 5367), 5′-TCAAGCAGTTGACACTTGACTGTG-3′ (SEQ ID NO:37) and 5′-TAATCTCACAGTGATGAGAGGAGA-3′ (SEQ ID NO:38), amplicon size 260 bp; 296S, 5′-CTGTGTCTCAATCTTGAACAAACACA-3′ (SEQ ID NO:39) and 5′-GGAAGAGAGACAGTAAACATTTCGT-3′ (SEQ ID NO:40), amplicon size 266 bp; 331T, 5′-CTCCCTTCCTTCCTGATCGTTTTC-3′ (SEQ ID NO:41) and 5′-CGGCTCTCAAGCACTGCAGATTTTG-3′ (SEQ ID NO:42), amplicon size 111 bp. The marker 296S was derived from the end sequence of a BAC clone containing the Nope gene and is located 5′-upstream of Nope; the marker 331T was derived from an overlapping BAC clone containing part of the Nope gene and all of the Punc gene (Salbaum, supra, 1999) and is located 3′-downstream of the Punc gene. Hot start PCR conditions were 94° C. for 3 min, followed by 35 cycles of 93° C. for 30 seconds, 58° C. for 30 seconds, and 72° C. for 75 seconds, and finished at 72° C. for 5 minutes. Assays for each primer pair were performed in triplicate, analyzed by agarose gel electrophoresis, and summarized as data vectors. Results were analyzed using on-line mapping software provided by the Whitehead Institute Center for Genome Research (http://www.genome.wi.mit.edu/mouse_rh/index.html), which allowed placement of all markers relative to framework markers on mouse chromosome 9.

PCR assays with primer pairs specific for each gene or marker were carried out using DNA from the T31 panel. The results of the PCR assays were summarized as data vectors, which are shown in Table 1. “1” indicates the presence, “O” indicates the absence, and “2” indicates the weak presence of a PCR product. The T31 panel shown in Table 1 represents 100 cell lines in which fragments of irradiated mouse genomic DNA were introduced by fusion with hamster cells. The total panel represents the whole mouse genome. Based on the pattern of hybridization and comparison to known markers, Nope was found to map to chromosome 9.

TABLE 1

| Gene/Marker | T31 data vector: |
|---|---|
| Punc | 00000 01210 20002 00000 00020 01010 20000 00001 10000 00102 10000 10101 02010 |
| | 00010 10000 10001 10000 00000 01001 00001 |
| Nope | 00000 01210 00002 00000 00020 01010 10010 00001 10000 00101 10000 10101 00010 |
| | 00210 10000 10001 10002 00200 01001 00001 |
| Ngn | 00000 00010 00110 00000 00020 01010 10000 00001 10000 00102 00010 20101 00010 |
| | 00200 00010 10101 11000 00000 02101 00000 |
| 331T | 00000 01110 00002 00000 00020 01020 20020 00001 10000 00101 10000 00001 00210 |
| | 02000 00000 00000 00000 00000 00000 00000 |
| 296S | 00000 01210 00002 01000 00020 01012 10020 00021 10000 00102 10000 10101 00020 |
| | 00210 00000 20002 20000 00000 02002 00001 |

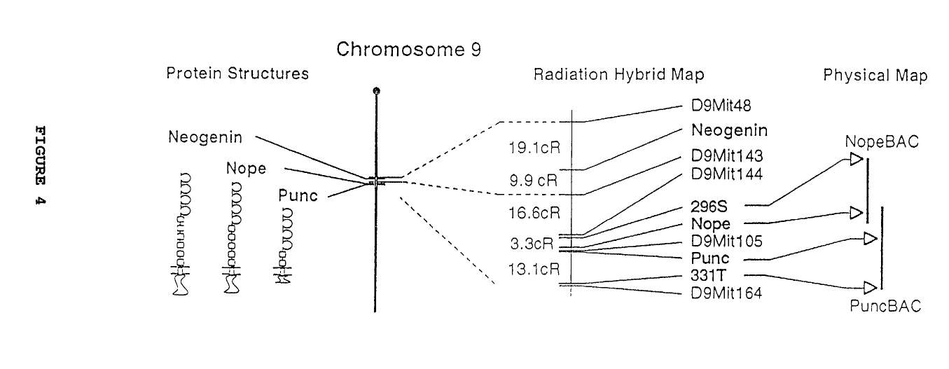

Neogenin maps very close to both Nope and Punc on chromosome 9 (FIG. 4). Localization of the genes for Nope, Punc and Neogenin on mouse chromosome 9 are shown in FIG. 4. Structures of the encoded proteins are indicated next to the chromosome sketch. Placement of Neogenin, Nope, Punc, and BAC end markers relative to framework markers D9Mit48 and D9Mit143 on chromosome 9 are indicated. Distances are given in cR. The arrangement of BAC clones and the origin of PCR products used for mapping is shown on the right.

Neogenin showed linkage to the framework marker D9Mit48 at 31.7 cM, whereas Nope, Punc, and both BAC end markers were found to be linked to the framework marker D9Mit143, which is also placed at 31.7 cM. Neogenin therefore maps to a region of mouse chromosome 9 that is syntenic to the region on human chromosome 15 where Neogenin had been place on the cytogenetic map. Radiation hybrid mapping of Nope and Punc confirmed previous cytogenetic mapping experiments and established a link between the radiation hybrid and cytogenetic map of mouse chromosome 9.

Based on the similarity to DCC, Punc and Nope are members of a subgroup of the DCC Ig superfamily, termed herein DEAL, for DCC et al. The high sequence similarity between Punc and Nope, identical direction of transcription, and close physical proximity initially suggested that these two genes likely arose through a gene duplication event. The mapping data place the gene for Neogenin, a core member of the Deal subgroup, in the immediate vicinity of the genes for Nope and Punc. These genes are therefore clustered on chromosome 9. In light of the finding that C. elegans, and, as of this time Drosophila, have only one DEAL gene in their respective genomes, it is likely that the gene cluster on chromosome 9 in the mouse is the result of a recent linear expansion of the DEAL family that occurred only in the vertebrate lineage. Given the closer relationship of DCC and Neogenin to the invertebrate counterparts, Neogenin represents a more ancestral gene in the cluster, and Nope and Punc are more derived. If so, the divergence of Punc and Nope included sequence variation and domain loss, possibly through intragenic recombination events. Further mapping in this region of chromosome 9 will reveal whether there are additional members of this gene cluster.

In contrast to the structural similarities between these DEAL genes on chromosome 9, expression is strikingly different. The Punc gene is expressed shortly after gastrulation in the neuroectoderm, and its expression domain includes early proliferating cells in the developing nervous system and the lateral plate mesoderm. After mid-gestation, Punc undergoes a down regulation that appears unusual for Ig CAMs. In comparison to Punc, Nope expression increases as development progresses and persists in the adult animal. Such an overall pattern is more commonly observed with Ig superfamily genes and typically attributed to the fact that cell surface properties of differentiated cells are more elaborate compared to proliferating precursor cells. Nope and Punc differ not only in their temporal but also in their cellular specificity of expression, suggesting that divergence of genes in this region is not restricted to coding regions but includes regulatory elements. This latter point is of particular interest given the close proximity of these two genes. Identification of regulatory sequences of Nope and Punc is useful for understanding the specificity of expression of these genes in developmental gene regulation.

The 3′-untranslated region of the Nope gene showed sequence homology to two human STS markers, WI-18508 and WI-16786 (Salbaum, supra, 1999). Both markers have been mapped close to a locus on a chromosome 15 which is linked to Bardet-Biedl syndrome 4 (Carmi et al., Hum. Mol. Genet. 4:9-13 (1995)). Considering that mental retardation is part of Bardet-Biedl syndrome, it was intriguing to detect Nope gene expression in the adult hippocampus, an area of the brain associated with cognitive functions such as learning and memory. Therefore, it is possible that the Nope gene plays a role in related human disorders.

Throughout this application various publications have been referenced. The disclosures of these publications in their entireties are hereby incorporated by reference in this application in order to more fully describe the state of the art to which this invention pertains.

Although the invention has been described with reference to the examples provided above, it should be understood that various modifications can be made without departing from the spirit of the invention. Accordingly, the invention is limited only by the claims.

I claim:

1. An isolated Nope polypeptide, or functional fragment thereof, comprising the amino acid sequence of a Nope polypeptide (SEQ ID NO:2), or a modification thereof.

2. The isolated Nope polypeptide of claim 1, wherein said functional fragment comprises the amino acid sequence of a Nope polypeptide extracellular domain (SEQ ID NO:4).

3. The isolated Nope polypeptide of claim 2, wherein said functional fragment comprises an amino acid sequence selected from the group consisting of immunoglobulin domain 1 (SEQ ID NO:8), immunoglobulin domain 2 (SEQ ID NO:10), immunoglobulin domain 3 (SEQ ID NO:12), immunoglobulin domain 4 (SEQ ID NO:14), fibronectin domain 1 (SEQ ID NO:16), fibronectin domain 2 (SEQ ID NO:18), fibronectin domain 3 (SEQ ID NO:20), fibronectin domain 4 (SEQ ID NO:22), and fibronectin domain 5 (SEQ ID NO:24).

4. The isolated Nope polypeptide of claim 1, wherein said functional fragment comprises the amino acid sequence of a Nope polypeptide intracellular domain (SEQ ID NO:6).

5. An antibody that specifically binds the Nope polypeptide of claim 1.

6. The antibody of claim 5, wherein said antibody is a polyclonal antibody.

7. The antibody of claim 5, wherein said antibody is a monoclonal antibody.

8. A method of detecting a Nope polypeptide, comprising contacting a sample with the antibody of claim 5, and detecting specific binding of said antibody.

9. An isolated nucleic acid molecule encoding a Nope polypeptide amino acid sequence referenced as SEQ ID NO:2, or a modification thereof.

10. An isolated nucleic acid molecule comprising the nucleotide sequence referenced as SEQ ID NO:1, or a modification thereof.

11. The nucleic acid molecule of claim 10, wherein said nucleotide sequence is selected from the group consisting of SEQ ID NOS:3, 5, 7, 9, 11, 13, 15, 17, 19, 21 and 23.

12. A Nope oligonucleotide, comprising between 15 and 300 contiguous nucleotides of SEQ ID NO:1 or the anti-sense strand thereof.

13. The isolated Nope oligonucleotide of claim 12, wherein said oligonucleotide comprises between 15 and 300 contiguous nucleotides of SEQ ID NO:5 or the anti-sense strand thereof.

14. A vector comprising an expression element operationally linked to the nucleotide sequence of claim 10.

15. A host cell comprising the vector of claim 13.

16. A method of detecting a Nope nucleic acid molecule in a sample, comprising contacting said sample with a Nope oligonucleotide of claim 12 under conditions allowing specific hybridization to a Nope nucleic acid molecule, and detecting said specific hybridization.

17. A method of detecting a Nope nucleic acid molecule in a sample, comprising contacting said sample with a Nope oligonucleotide of claim 13 under conditions allowing specific hybridization to a Nope nucleic acid molecule, and detecting said specific hybridization.

18. A method of detecting a Nope nucleic acid molecule in a sample, comprising contacting said sample with two or more Nope oligonucleotides of claim 12, amplifying a nucleic acid molecule, and detecting said amplification.

19. The method of claim 18, wherein said amplification is performed using polymerase chain reaction.

20. A kit comprising one or more Nope oligonucleotides comprising between 15 and 300 contiguous nucleotides of SEQ ID NO:1 or the anti-sense strand thereof.
